SEQUENCE LISTING (35 sequences)

SEQ ID NO: 1 - protein, 447 aa, Artificial sequence
  anti-PD-1 (pembrolizumab) heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 218 aa, Artificial sequence
  anti-PD-1 (pembrolizumab) light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 440 aa, Artificial sequence
  anti-PD-L1 (nivolumab) heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 214 aa, Artificial sequence
  anti-PD-L1 (nivolumab) light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, Artificial sequence
  anti-CTLA4 (ipilimumab) heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 215 aa, Artificial sequence
  anti-CTLA4 (ipilimumab) light chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 468 aa, Artificial sequence
  anti-TNFa (adalimumab) heavy chain
MGVKVLFALICIAVAEAEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 213 aa, Artificial sequence
  anti-TNFa (adalimumab) light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 453 aa, Artificial sequence
  anti-VEGF (bevacizumab) heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial sequence
  anti-VEGF (bevacizumab) light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 127 aa, Artificial sequence
  anti-PD-1 nanobody
QVQLQESGGGLVQPGGSLRLSCAASGFTSRNYAMTWVRQAPEKGLEWVSSISSDDDSTYYEYSVKGRFTISRDNAKNTLYLQLNSLKTEDTAMYYCTKEFVAVVPVLKLGRPRDLGQGTQVTVSSAA

SEQ ID NO: 12 - protein, 127 aa, Artificial sequence
  anti-PD-L1 nanobody
QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVS

SEQ ID NO: 13 - protein, 117 aa, Artificial sequence
  anti-CTLA4 nanobody
QVQLQESGGGSVQAGGSLRLSCTASGFGVDGTDMGWYRQAPGNECELVSSISSIGIGYYSESVKGRFTISRDNAKNTVYLQMNSLRPDDTAVYYCGRRWIGYRCGNWGRGTQVTVSS

SEQ ID NO: 14 - protein, 135 aa, Artificial sequence
  anti-TNFa nanobody
MGSQVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSSAGA

SEQ ID NO: 15 - protein, 126 aa, Artificial sequence
  anti-VEGF nanobody
QVQLQESGGGSLQAGASLRLSCAASGFAYSTYSMGWFRQVSGKEREGVATINSGTFRLWYTDSVKGSFTISRDNAKNMLYLQMNSLKPEDTAIYYCAARAWSPYSSTVDAGDFRYWGQGTQVTVSS

SEQ ID NO: 16 - protein, 236 aa, Mus musculus
MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 17 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 18 - protein, 211 aa, Mus musculus
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 19 - protein, 140 aa, Mus musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 20 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 21 - protein, 153 aa, Homo sapiens [sequence omitted]

SEQ ID NO: 22 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 23 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 24 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 25 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 26 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - protein, 154 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 28 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 29 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - protein, 162 aa, Mus musculus
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 31 - protein, 153 aa, Homo sapiens; identical to SEQ ID NO: 26

SEQ ID NO: 32 - protein, 190 aa, Artificial sequence
  Igis_Sig/mIL-2
METDTLLLWVLLLWVPGSTGDMYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 33 - protein, 169 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 34 - protein, 153 aa, Homo sapiens; identical to SEQ ID NO: 26

SEQ ID NO: 35 - protein, 594 aa, Artificial sequence
  hyPBase
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

(19) United States
(12) Patent Application Publication
(10) Pub. No.: US 2023/0210908 A1
(43) Pub. Date: Jul. 6, 2023

(54) METHODS FOR IMPROVED DELIVERY OF THERAPEUTIC AGENTS

(71) Applicant: William Marsh Rice University, Houston, TX (US)

(72) Inventors: Omid VEISEH, Houston, TX (US); Amanda NASH, Houston, TX (US); Bhagyashree Kishor BACHHAV, Houston, TX (US); Carlos Alberto ORIGEL MARMOLEJO, Houston, TX (US); Damon BERMAN, Houston, TX (US); Christian SCHREIB, Houston, TX (US); Laura SEGATORI, Houston, TX (US); Alen TRUBELJA, Houston, TX (US); Oleg A. IGOSHIN, Houston, TX (US); Andrew D. HECHT, Houston, TX (US)

(73) Assignee: William Marsh Rice University, Houston, TX (US)

(21) Appl. No.: 17/904,106

(22) PCT Filed: Feb. 11, 2021
(86) PCT No.: PCT/US2021/017535

§ 371 Date: Aug. 11, 2022

Related U.S. Application Data
(60) Provisional application No. 62/972,944, filed on Feb. 11, 2020.

(51) Int. Cl. A61K 35/36; C12N 5/071; C12N 15/90; C12N 15/63; C07K 14/715; C07K 14/55

(52) U.S. Cl. CPC A61K 35/36; C07K 14/55; C07K 14/7155; C12N 5/0686; C12N 15/635; C12N 15/907; A61K 38/00; C12N 2830/001; C12N 2840/203

(57) ABSTRACT

The present disclosure provides expression constructs designed to provide for expression of therapeutic proteins from engineered cells. The engineered cells may be encapsulated into implantable elements that allow for the therapeutic protein to be released into from the capsule while protecting the cell from the immune system of a patient into which the capsule is implanted.

### REFERENCE TO RELATED APPLICATIONS

The present application claims the priority benefit of U.S. provisional application no. 62/972,944, filed Feb. 11, 2020, the entire contents of which is incorporated herein by reference.

### STATEMENT REGARDING FEDERALLY SPONSORED RESEARCH

This invention was made with government support under Grant No. R01DK120459 awarded by the National Institutes of Health, Grant Nos. MCB-1615562 and CBET-1805317 awarded by the National Science Foundation, Grant Nos. HR001119S0027 and N6600119C4020 awarded by the Department of Defense. The government has certain rights in the invention.

### REFERENCE TO A SEQUENCE LISTING

The instant application contains a Sequence Listing which has been submitted electronically in ASCII format and is hereby incorporated by reference in its entirety. The ASCII copy, created on Feb. 8, 2021, is named RICEP0069WO_ST25 and is 74.0 kilobytes in size.

### BACKGROUND

The development of this disclosure was funded in part by the Cancer Prevention and Research Institute of Texas (CPRIT) under Grant No. RR160047 and by the Welch Foundation under Grant No. C-1995.

### 1. Field

The present disclosure relates generally to the fields of biology, medicine, bioengineering, and cell encapsulation. More particularly, it concerns compositions and methods for delivery of biologic molecules of a variety of sizes and functions. The methods involve cell engineering as well as biomaterials synthesis.

### 2. Description of Related Art

Monoclonal antibodies are one of the best-selling classes of biopharmaceuticals. However, there is a lack of technology that integrates long-term production of stable antibodies or cytokines in a hydrogel device that prevents immune attack using immunomodulatory drugs on the exterior shell of the device.

### SUMMARY

Provided herein are compositions of engineered cells that are encapsulated into a core-shell immunomodulatory alginate. These compositions provide for adaptive and programmable sustained delivery of various biologic and therapeutic molecules, such as cytokines or monoclonal antibodies, for cancer immunotherapy or auto-immune disorders.

In one embodiment, provided herein are engineered cells, or implantable elements comprising the engineered cells, wherein the engineered cells comprise an exogenous nucleic acid having a coding sequence encoding a therapeutic protein. In some aspects, the exogenous nucleic acid is integrated into a chromosome of the engineered cells. In some aspects, the therapeutic protein is an antibody or a cytokine.

In some aspects, the therapeutic protein is an antibody. In some aspects, the antibody’s heavy chain and the antibody’s light chain are expressed by two different open reading frames operably linked to two different promoters. In some aspects, both promoters are strong, constitute promoters in the engineered cell. In some aspects, each of the open reading frames is present on a separate exogenous nucleic acid. In some aspects, each of the open reading frames is present on the same exogenous nucleic acid. In some aspects, the heavy chain and the light chain are expressed in a single open reading frame with the coding sequences for each chain being separated by an internal ribosome entry site. In some aspects, the promoter is a strong, constitutive promoter in the engineered cell.

In some aspects, the engineered cell further comprises at least one coding sequence encoding a selection marker. In some aspects, the selection marker is an antibiotic resistance gene. In some aspects, a coding sequence encoding the selection marker is present on each exogenous nucleic acid the comprises a coding sequence encoding a therapeutic protein. In some aspects, the coding sequence encoding the selection marker is operably linked to a separate promoter from the promoter that is operably linked to the coding sequence encoding the therapeutic protein. In some aspects, the coding sequence encoding the selection marker is operably linked to the same promoter as the coding sequence encoding the therapeutic protein. In some aspects, the coding sequence encoding the selection marker and the coding sequence encoding the therapeutic protein are separated by an internal ribosomal entry site. In some aspects, the antibody is a anti-PD-1, anti-PD-L1, anti-CTLA4, anti-TNFα, or anti-VEGF antibody.

In some aspects, the therapeutic protein is a cytokine. In some aspects, the cytokine is IL-1, IL,-1α, II,-1β, IL,-1RA, IL-2, IL-4, IL-5, IL-6, IL-7, IL-8, IL-9, IL-10, IL-11, IL-12, IL-12a, IL-12b, IL-13, IL-14, IL-16, IL-17, G-CSF, GM-CSF, IL-20, IFN-α, IFN-β, IFN-y, CD154, LT-β, CD70, CD153, CD178, TRAIL, TNF-α, TNF-β, SCF, M-CSF, MSP, 4-1BBL, LIF, or OSM. In some aspects, the cytokine is IL-2.

In some aspects, the cytokine coding sequence is operably linked to a repressible promoter. In some aspects, the engineered cells further comprises at least one coding sequence encoding a transcriptional repressor that can bind to the repressible promoter, wherein the transcriptional repressor coding sequence is operably linked to a promoter that is activated as a result of signaling through the cytokine’s receptor. In some aspects, the cytokine coding sequence comprises a translation regulatory higher-order structure in its 5′ untranslated region. In some aspects, the engineered cell further comprises at least one coding sequence encoding an RNA-binding translation repressor that can bind to the higher-order structure, wherein the RNA-binding translation repressor coding sequence is operably linked to a promoter that is activated as a result of signaling through the cytokine’s receptor. In some aspects, the cytokine coding sequence comprises one or more miRNA binding sites in its 3′ untranslated region. In some aspects, the engineered cell further comprises at least one coding sequence encoding an miRNA that can bind to the miRNA binding sites, wherein the miRNA coding sequence is operably linked to a promoter that is activated as a result of signaling through the cytokine’s receptor. In some aspects, the engineered cell further comprises at least one coding sequence encoding a ubiquitin ligase that can bind to the cytokine, wherein the ubiquitin ligase coding sequence is operably linked to a promoter that is activated as a result of signaling through the cytokine’s receptor. In some aspects, the cytokine coding sequence is operably linked to a small molecule-activated promoter. In some aspects, the cytokine coding sequence comprises an activating or inhibiting small molecule-dependent functional higher-order structure. In some aspects, the cytokine coding sequence comprises a small molecule-assisted shutoff system sequence. In some aspects, the cytokine coding sequence is operably linked to a synthetic promoter that is activated by a synthetic transcription factor. In some aspects, the synthetic transcription factor comprises a catalytically inactive Cas9 (dCas9) fused to transcriptional activation domains. In some aspects, the synthetic transcription factor coding sequence is operably linked to a small molecule-activated promoter. In some aspects, the synthetic transcription factor coding sequence comprises an activating or inhibiting small molecule-dependent functional higher-order structure. In some aspects, the synthetic transcription factor coding sequence comprises a small molecule-assisted shutoff system sequence.

In some aspects, the production of a cytokine from a cytokine-producing cell (e.g., an IL-2 producing RPE cell) is regulated in response to the level of a second component. In some aspects, the second component may be a protein, such as interferon-y (IFN-γ). In some aspects, a degradation event, e.g., apoptosis, is triggered in the cytokine-producing cell (e.g., the IL-2 producing RPE cell) upon detection of the second component (e.g., a protein, e.g., IFN-γ), e.g., detection of a level of the second component (e.g., a threshold level).

In some aspects, the present disclosure further comprises a method of modeling a feature of the feedback loop. For example, the method of modeling (e.g., an algorithm) may be used to predict the timing of an event in the feedback loop, e.g., the time delay between detection of the second component (e.g., a protein, e.g., IFN-γ) and initiation of the apoptotic pathway may vary in length.

In some embodiments, control of the feedback loop comprises expression of a transcriptional repressor in response to a target gene. In some embodiments, the transcriptional repressor is EKRAB. In some embodiments, the target gene is an IFN-γ response gene (e.g., RPE65). In some embodiments, a pro-apoptotic gene is expressed under control of the transcriptional repressor. In some embodiments, the pro-apoptotic gene is bax.

In some aspects, the engineered cell expresses more than one therapeutic protein. In some aspects, the engineered cell expresses three therapeutic proteins. In some aspects, the engineered cell expresses four therapeutic proteins.

In some aspects, the engineered cell is a Chinese hamster ovary (CHO) cell, human embryonic kidney (HEK) cell, retinal pigmented epithelium (ARPE-10) cell, mesenchymal stem cell (MSC), human umbilical vein endothelial cell (HUVEC), murine myeloma NS0 and Sp2/0 cell, BABL/3T3 cell, MDCK cell, or PER.C6 cell.

In some aspects, the exogenous nucleic acid is an expression vector. In some aspects, the expression vector is pcDNA3.1. In some aspects, the exogenous nucleic acid is a viral vector. In some aspects, the viral vector is a lentiviral vector.

In some aspects, the exogenous nucleic acid is a transposon system. In some aspects, the transposon system is a piggyBac expression system.

In some aspects, the engineered cell further comprises an exogenous nucleic acid having a coding sequence encoding a kill switch. In some aspects, the kill switch is chimeric caspase-9 fused to a rimiducid-induced switch.

In some aspects, the engineered cell is further engineered to increase its immunogenicity. In some aspects, the engineered cell releases the therapeutic protein.

In some aspects, the implantable element comprises an inner zone and an outer zone, wherein the engineered cell is present in the inner zone. In some aspects, the outer zone is configured so as to hinder contact of a host immune effector molecule or cell with the antigenic agent for an initial or shielded phase of implantation, but so as to allow contact of a host immune effector molecule or cell with the antigenic agent in a subsequent or unshielded phase of implantation. In some aspects, the outer zone comprises a degradable entity. In some aspects, the shielded phase lasts for no longer than 1 hour, 12 hours, 1 day, 2 days, 3 days, 6 days, or 12 days. In some aspects, the shielded phase lasts for between 0.5 days and 30 days, 1 day and 14 days, and 1 day and 7 days. In some aspects, the thickness of the outer zone correlates with the length/duration of the shielded phase.

In some aspects, the implantable construct provides sustained release of the therapeutic protein. In some aspects, the implantable construct provides substantially non-pulsatile release of the therapeutic protein. In some aspects, the implantable element further comprises a polymeric hydrogel. In some aspects, the outer zone comprises a polymeric hydrogel. In some aspects, the inner zone comprises a polymeric hydrogel. In some aspects, the inner zone and the outer zone comprise the same polymeric hydrogel. In some aspects, the inner zone and the outer zone comprise two different polymeric hydrogels. In some aspects, the polymeric hydrogel comprises chitosan, cellulose, hyaluronic acid, or alginate.

In some aspects, the implantable element comprises an engineered cell that produces a single type of therapeutic protein. In some aspects, the implantable element comprises an engineered cell that produces a plurality of therapeutic proteins. In some aspects, the implantable element comprises a first engineered cell and a second engineered cell that each produces a different therapeutic protein. In some aspects, the first engineered cell produces a first therapeutic antibody and the second engineered cell produces a second therapeutic protein.

In some aspects, the implantable element comprises at least about 10,000, 15,000, or 20,000 engineered cells. In some aspects, the implantable element further comprises an additional therapeutic agent. In some aspects, the additional therapeutic agent is a chemotherapeutic agent or an immunomodulatory agent.

In one embodiment, provided herein are bioreactors comprising the engineered cells of any one of the present embodiments. In one embodiment, provided herein are preparations of implantable elements comprising a plurality of implantable elements of any one of the present embodiments. In some aspects, the preparation is a pharmaceutically acceptable preparation.

In one embodiment, provided herein are methods of providing an implantable element to a patient, the method comprising implanting into the subject, or providing the subject with, an implantable element of any one of the present embodiments. In some aspects, the method treats the patient for a disorder that comprises unwanted cell proliferation.

In one embodiment, provided herein are methods of administering an immune checkpoint inhibitor to a patient having a cancer, the method comprising implanting into the intraperitoneal space of the patient an implantable element of any one of the present embodiments, wherein the implantable element is configured to release the immune checkpoint inhibitor. In some aspects, the immune checkpoint inhibitor is a PD-L1 antibody, a PD-1 antibody, or a CTLA4 antibody. In some aspects, the methods further comprise administering an anti-cancer therapy to the patient. In some aspects, the anti-cancer therapy is a surgical therapy, a chemotherapy, a radiation therapy, a cryotherapy, a hormonal therapy, a toxin therapy, an immunotherapy, or a cytokine therapy. In some aspects, the cancer is a colorectal cancer, a neuroblastoma, a breast cancer, a pancreatic cancer, a brain cancer, a lung cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

In one embodiment, provided herein are methods of treating a cancer in a patient, the method comprising implanting into the intraperitoneal space of the patient an implantable element of any one of the present embodiments, wherein the implantable element is configured to release the therapeutic protein at a level sufficient to promote immune effector cell-mediated attack on the cancer but not great enough to promote Treg levels in the cancer. In some aspects, the therapeutic protein is an immune checkpoint inhibitor. In some aspects, the immune checkpoint inhibitor is a PD-L1 antibody, a PD-1 antibody, or a CTLA4 antibody. In some aspects, the methods further comprise administering an anti-cancer therapy to the patient. In some aspects, the anti-cancer therapy is a surgical therapy, a chemotherapy, a radiation therapy, a cryotherapy, a hormonal therapy, a toxin therapy, an immunotherapy, or a cytokine therapy. In some aspects, the cancer is a colorectal cancer, a neuroblastoma, a breast cancer, a pancreatic cancer, a brain cancer, a lung cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

The disclosure is further described in the following numbered embodiments:
1. An engineered cell, or an implantable element comprising the engineered cell, wherein the engineered cell comprises an exogenous nucleic acid having a coding sequence encoding a therapeutic protein.
2. The engineered cell or implantable element comprising the engineered cell of embodiment 1, wherein the exogenous nucleic acid is integrated into a chromosome of the engineered cell.
3. The engineered cell or implantable element comprising the engineered cell of embodiment 1 or 2, wherein the therapeutic protein is an antibody or a cytokine.
4. The engineered cell or implantable element comprising the engineered cell of embodiment 3, wherein the therapeutic protein is an antibody.
5. The engineered cell or implantable element comprising the engineered cell of embodiment 4, wherein the antibody’s heavy chain and the antibody’s light chain are expressed by two different open reading frames operably linked to two different promoters.
6. The engineered cell or implantable element comprising the engineered cell of embodiment 5, wherein both promoters are strong, constitute promoters in the engineered cell.
7. The engineered cell or implantable element comprising the engineered cell of embodiment 5 or 6, wherein each of the open reading frames is present on a separate exogenous nucleic acid.
8. The engineered cell or implantable element comprising the engineered cell of embodiment 5 or 6, wherein each of the open reading frames is present on the same exogenous nucleic acid.
9. The engineered cell or implantable element comprising the engineered cell of embodiment 4, wherein the heavy chain and the light chain are expressed in a single open reading frame with the coding sequences for each chain being separated by an internal ribosome entry site.
10. The engineered cell or implantable element comprising the engineered cell of embodiment 9, wherein the promoter is a strong, constitutive promoter in the engineered cell.
11. The engineered cell or implantable element comprising the engineered cell of any one of embodiments 1-10, wherein the engineered cell further comprises at least one coding sequence encoding a selection marker.
12. The engineered cell or implantable element comprising the engineered cell of embodiment 11, wherein the selection marker is an antibiotic resistance gene.
13. The engineered cell or implantable element comprising the engineered cell of embodiment 11 or 12, wherein a coding sequence encoding the selection marker is present on each exogenous nucleic acid the comprises a coding sequence encoding a therapeutic protein.
14. The engineered cell or implantable element comprising the engineered cell of embodiment 13, wherein the coding sequence encoding the selection marker is operably linked to a separate promoter from the promoter that is operably linked to the coding sequence encoding the therapeutic protein.
15. The engineered cell or implantable element comprising the engineered cell of embodiment 13, wherein the coding sequence encoding the selection marker is operably linked to the same promoter as the coding sequence encoding the therapeutic protein.
16. The engineered cell or implantable element comprising the engineered cell of embodiment 15, wherein the coding sequence encoding the selection marker and the coding sequence encoding the therapeutic protein are separated by an internal ribosomal entry site.
17. The engineered cell or implantable element comprising the engineered cell of any one of embodiments 4-16, wherein the antibody is a anti-PD-1, anti-PD-L1, anti-CTLA4, anti-TNFα, or anti-VEGF antibody.
18. The engineered cell or implantable element comprising the engineered cell of embodiment 3, wherein the therapeutic protein is a cytokine.
19. The engineered cell or implantable element comprising the engineered cell of embodiment 18, wherein the cytokine is IL-1, IL-1α, IL-1β, IL-1RA, IL-2, IL-4, IL-5, IL-6, IL-7, IL-8, IL-9, IL-10, IL-11, IL-12, IL-12a, IL-12b, IL-13, IL-14, IL-16, IL-17, G-CSF, GM-CSF, IL-20, IFN-α, IFN-β, IFN-γ, CD154, LT-β, CD70, CD153, CD178, TRAIL, TNF-α, TNF-β, SCF, M-CSF, MSP, 4-1BBL, LIF, or OSM.
20. The engineered cell or implantable element comprising the engineered cell of embodiment 18 or 19, wherein the cytokine coding sequence is operably linked to a repressible promoter.
21. The engineered cell or implantable element comprising the engineered cell of embodiment 20, wherein the engineered cell further comprises at least one coding sequence encoding a transcriptional repressor that can bind to the repressible promoter, wherein the transcriptional repressor coding sequence is operably linked to a promoter that is activated as a result of signaling through the cytokine’s receptor.
22. The engineered cell or implantable element comprising the engineered cell of embodiment 18 or 19, wherein the cytokine coding sequence comprises a translation regulatory higher-order structure in its 5′ untranslated region.
23. The engineered cell or implantable element comprising the engineered cell of embodiments 22, wherein the engineered cell further comprises at least one coding sequence encoding an RNA-binding translation repressor that can bind to the higher-order structure, wherein the RNA-binding translation repressor coding sequence is operably linked to a promoter that is activated as a result of signaling through the cytokine’s receptor.
24. The engineered cell or implantable element comprising the engineered cell of embodiment 18 or 19, wherein the cytokine coding sequence comprises one or more miRNA binding sites in its 3′ untranslated region.
25. The engineered cell or implantable element comprising the engineered cell of any one of embodiments 20-24, wherein the production of the cytokine is regulated in response to the level of a second component.
26. The engineered cell or implantable element comprising the engineered cell of embodiment 25, wherein the second component is a protein, e.g., IFN-γ.
27. The engineered cell or implantable element comprising the engineered cell of embodiment 25 or 26, wherein detection of the second component triggers a degradation event (e.g., apoptosis) in the cell.
28. The engineered cell or implantable element comprising the engineered cell of embodiment 24, wherein the engineered cell further comprises at least one coding sequence encoding an miRNA that can bind to the miRNA binding sites, wherein the miRNA coding sequence is operably linked to a promoter that is activated as a result of signaling through the cytokine’s receptor.
29. The engineered cell or implantable element comprising the engineered cell of embodiment 18 or 19, wherein the engineered cell further comprises at least one coding sequence encoding a ubiquitin ligase that can bind to the cytokine, wherein the ubiquitin ligase coding sequence is operably linked to a promoter that is activated as a result of signaling through the cytokine’s receptor.
30. The engineered cell or implantable element comprising the engineered cell of any one of embodiments 18-29, wherein the cytokine coding sequence is operably linked to a small molecule-activated promoter.
31. The engineered cell or implantable element comprising the engineered cell of any one of embodiments 18-30, wherein the cytokine coding sequence comprises an activating or inhibiting small molecule-dependent functional higher-order structure.
32. The engineered cell or implantable element comprising the engineered cell of any one of embodiments 18-31, wherein the cytokine coding sequence comprises a small molecule-assisted shutoff system sequence.
33. The engineered cell or implantable element comprising the engineered cell of any one of embodiments 18-31, wherein the cytokine coding sequence is operably linked to a synthetic promoter that is activated by a synthetic transcription factor.
34. The engineered cell or implantable element comprising the engineered cell of embodiment 33, wherein the synthetic transcription factor comprises a catalytically inactive Cas9 (dCas9) fused to transcriptional activation domains.
35. The engineered cell or implantable element comprising the engineered cell of embodiment 33 or 34, wherein the synthetic transcription factor coding sequence is operably linked to a small molecule-activated promoter.
36. The engineered cell or implantable element comprising the engineered cell of any one of embodiments 33-35, wherein the synthetic transcription factor coding sequence comprises an activating or inhibiting small molecule-dependent functional higher-order structure.
37. The engineered cell or implantable element comprising the engineered cell of any one of embodiments 33-36, wherein the synthetic transcription factor coding sequence comprises a small molecule-assisted shutoff system sequence.
38. The engineered cell or implantable element comprising the engineered cell of any one of embodiments 1-19, wherein the engineered cell expresses more than one therapeutic protein.
39. The engineered cell or implantable element comprising the engineered cell of any one of embodiments 1-38, wherein the engineered cell expresses three therapeutic proteins.
40. The engineered cell or implantable element comprising the engineered cell of any one of embodiments 1-38, wherein the engineered cell expresses four therapeutic proteins.
41. The engineered cell or implantable element comprising the engineered cell of any one of embodiments 1-40, wherein the engineered cell is a Chinese hamster ovary (CHO) cell, human embryonic kidney (HEK) cell, retinal pigmented epithelium (ARPE-10) cell, mesenchymal stem cell (MSC), human umbilical vein endothelial cell (HUVEC), murine myeloma NS0 and Sp2/0 cell, BABL/3T3 cell, MDCK cell, or PER.C6 cell.
42. The engineered cell or implantable element comprising the engineered cell of any one of embodiments 1-41, wherein the exogenous nucleic acid is an expression vector.
43. The engineered cell or implantable element comprising the engineered cell of embodiment 42, wherein the expression vector is pcDNA3.1.
44. The engineered cell or implantable element comprising the engineered cell of any one of embodiments 1-41, wherein the exogenous nucleic acid is a viral vector.
45. The engineered cell or implantable element comprising the engineered cell of embodiment 44, wherein the viral vector is a lentiviral vector.
46. The engineered cell or implantable element comprising the engineered cell of any one of embodiments 1-41, wherein the exogenous nucleic acid is a transposon system.
47. The engineered cell or implantable element comprising the engineered cell of embodiment 46, wherein the transposon system is a piggyBac expression system.
48. The engineered cell or implantable element comprising the engineered cell of any one of embodiments 1-47, wherein engineered cell further comprises an exogenous nucleic acid having a coding sequence encoding a kill switch.
49. The engineered cell or implantable element comprising the engineered cell of embodiment 48, wherein the kill switch is chimeric caspase-9 fused to a rimiducid-induced switch.
50. The engineered cell or implantable element comprising the engineered cell of any one of embodiments 1-49, wherein the engineered cell is further engineered to increase its immunogenicity.
51. The engineered cell or implantable element comprising the engineered cell of any one of embodiments 1-50, wherein the engineered cell releases the therapeutic protein.
52. The implantable element comprising the engineered cell of any one of embodiments 1-51, wherein the implantable element comprises an inner zone and an outer zone, wherein the engineered cell is present in the inner zone.
53. The implantable element comprising the engineered cell of embodiment 52, wherein the outer zone is configured so as to hinder contact of a host immune effector molecule or cell with the antigenic agent for an initial or shielded phase of implantation, but so as to allow contact of a host immune effector molecule or cell with the antigenic agent in a subsequent or unshielded phase of implantation.
54. The implantable element comprising the engineered cell of embodiment 52 or 53, wherein the outer zone comprises a degradable entity.
55. The implantable element comprising the engineered cell of embodiment 53, wherein the shielded phase lasts for no longer than 1 hour, 12 hours, 1 day, 2 days, 3 days, 6 days, or 12 days.
56. The implantable element comprising the engineered cell of embodiment 53, wherein the shielded phase lasts for between 0.5 days and 30 days, 1 day and 14 days, and 1 day and 7 days.
57. The implantable element comprising the engineered cell of any one of embodiments 53-56, wherein the thickness of the outer zone correlates with the length/duration of the shielded phase.
58. The implantable element comprising the engineered cell of any one of embodiments 1-57, wherein the implantable construct provides sustained release of the therapeutic protein.
59. The implantable element of any one of embodiments 1-57, wherein the implantable construct provides substantially non-pulsatile release of the therapeutic protein.
60. The implantable element of any one of embodiments 1-59, further comprising a polymeric hydrogel.
61. The implantable element of embodiment 60, wherein the outer zone comprises a polymeric hydrogel.
62. The implantable element of embodiment 60, wherein the inner zone comprises a polymeric hydrogel.
63. The implantable element of any one of embodiments 60-62, wherein the inner zone and the outer zone comprise the same polymeric hydrogel.
64. The implantable element of any one of embodiments 60-62, wherein the inner zone and the outer zone comprise two different polymeric hydrogels.
65. The implantable element of any one of embodiments 60-64, wherein the polymeric hydrogel comprises chitosan, cellulose, hyaluronic acid, or alginate.
66. The implantable element of any one of embodiments 1-65, wherein the implantable element comprises an engineered cell that produces a single type of therapeutic protein.
67. The implantable element of any one of embodiments 1-65, wherein the implantable element comprises an engineered cell that produces a plurality of therapeutic proteins.
68. The implantable element of any one of embodiments 1-65, wherein the implantable element comprises a first engineered cell and a second engineered cell that each produces a different therapeutic protein.
69. The implantable element of embodiment 68, wherein the first engineered cell produces a first therapeutic antibody and the second engineered cell produces a second therapeutic protein.
70. The implantable element of any one of embodiments 1-65, wherein the implantable element comprises at least about 10,000, 15,000, or 20,000 engineered cells.
71. The implantable element of any one of embodiments 1-70, wherein the implantable element further comprises an additional therapeutic agent.
72. The implantable element of any one of embodiments 1-71, wherein the additional therapeutic agent is a chemotherapeutic agent or an immunomodulatory agent.
73. A bioreactor comprising the engineered cell of any one of embodiments 1-51.
74. A preparation of implantable elements comprising a plurality of implantable elements of any one of embodiments 1-72.
75. The preparation of embodiment 74, wherein the preparation is a pharmaceutically acceptable preparation.
76. A method of providing an implantable element to a patient, the method comprising implanting into the subject, or providing the subject with, an implantable element of any one of embodiments 1-72.
77. The method of embodiment 76, wherein the method treats the patient for a disorder that comprises unwanted cell proliferation.
78. A method of administering an immune checkpoint inhibitor to a patient having a cancer, the method comprising implanting into the intraperitoneal space of the patient an implantable element of any one of embodiments 1-72, wherein the implantable element is configured to release the immune checkpoint inhibitor.
79. The method of embodiment 78, wherein the immune checkpoint inhibitor is a PD-L1 antibody, a PD-1 antibody, or a CTLA4 antibody.
80. The method of embodiment 78 or 79, further comprising administering an anti-cancer therapy to the patient.
81. The method of embodiment 80, wherein the anti-cancer therapy is a surgical therapy, a chemotherapy, a radiation therapy, a cryotherapy, a hormonal therapy, a toxin therapy, an immunotherapy, or a cytokine therapy.
82. The method of any one of embodiments 78-81, wherein the cancer is a colorectal cancer, a neuroblastoma, a breast cancer, a pancreatic cancer, a brain cancer, a lung cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.
83. A method of treating a cancer in a patient, the method comprising implanting into the intraperitoneal space of the patient an implantable element of any one of embodiments 1-72, wherein the implantable element is configured to release the therapeutic protein at a level sufficient to promote immune effector cell-mediated attack on the cancer but not great enough to promote Treg levels in the cancer.
84. The method of embodiment 83, wherein the therapeutic protein is an immune checkpoint inhibitor.
85. The method of embodiment 84, wherein the immune checkpoint inhibitor is a PD-L1 antibody, a PD-1 antibody, or a CTLA4 antibody.
86. The method of any one of embodiments 83-85, further comprising administering an anti-cancer therapy to the patient.
87. The method of embodiment 86, wherein the anti-cancer therapy is a surgical therapy, a chemotherapy, a radiation therapy, a cryotherapy, a hormonal therapy, a toxin therapy, an immunotherapy, or a cytokine therapy.
88. The method of any one of embodiments 83-87, wherein the cancer is a colorectal cancer, a neuroblastoma, a breast cancer, a pancreatic cancer, a brain cancer, a lung cancer, a stomach cancer, a skin cancer, a testicular cancer, a prostate cancer, an ovarian cancer, a liver cancer, an esophageal cancer, a cervical cancer, a head and neck cancer, a melanoma, or a glioblastoma.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 - Schematic of a single gene, dual vector system.

FIG. 2 - Schematic of a single vector, dual gene system.

FIG. 3 - Schematic of a bicistronic single ORF system.

FIG. 4 - Schematic of a tricistronic single ORF system.

FIG. 5 - Schematic of dual ORF auto-regulatory system using an operator/repressor.

FIG. 6 - Schematic of dual ORF auto-regulatory system using an RNA binding protein.

FIG. 7 - Schematic of dual ORF auto-regulatory system using a miRNA.

FIG. 8 - Schematic of dual ORF auto-regulatory system using a ubiquitin ligase.

FIG. 9 - Graph showing RPE levels in RPE cells treated with 0, 1, or 10 ng/mL recombinant human IFN-γ for 20 hours, monitored by RT-PCR.

FIG. 10 - Graph showing the variation of EKRAB and BAX degradation rates over time, allowing for a tunable delay before activation of apoptosis.

FIGS. 11A-B - Graphs showing the predicted pharmacokinetic models of IL-2 concentrations. FIG. 11A illustrates the predicted dynamics of IL-2 concentration over time in the intraperitoneal space and systemically, while FIG. 11B predicts the differences between the dose and the peak IL-2 concentrations over time.

FIG. 12 - Graph showing flow cytometry analyses of HEK293 cells expressing the expressing IL,-2αβy receptor transfected to express IL-2 and GFP under the control of a STAT5-inducible promoter (GFP, IL-2) and control cells lacking IL-2 (GFP).

FIGS. 13A-D - Schematic of four synthetic circuit topologies that execute repression of IL-2 production in response to STAT5 activation.

FIGS. 14A-C - Regulated cytokine circuit characterization. FIG. 14A is a graph showing normalized dose-response and response times (inset) of exemplary circuits described herein. FIG. 14B depicts exemplary equations for modeling. FIG. 14C is a graph for stimulated pharmacokinetics of intraperitoneal IL-2 concentrations for each circuit for a = 0.35. The dosage was scaled to ensure a similar peak IL-2 concentration. The insets show sensitivity analysis to production and cell death.

FIGS. 15A-D - Schematics of exemplary expression systems. FIG. 15A illustrates cassettes for expression of EKRAB, and FIGS. 15B-D illustrate cassettes for expression of IL-2 and fTA.

### DETAILED DESCRIPTION

Provided herein are engineered cells that stably express a molecule of interest. This is accomplished through transfection with an engineered plasmid to stably produce a number of nanobodies, cytokines, and antibodies, including anti-VEGF, anti-TNF-α, anti-PD-1, anti-PD-L1, and anti-CTLA4 antibodies, as a device for cancer immunotherapy, auto-immune disorder treatment, and industrial bioreactors.

A number of vector systems may be utilized for stable production of cytokines, nanobodies, or antibodies, including the piggybac transposon system, lentiviral vector, and the pcDNA3.1 vector system, thereby enabling their production in a number of mammalian cell lines. For example, in the case of antibody expression, the heavy chain and light chain may be expressed from two different vectors using two different promoters, with each vector having a selection marker. Alternatively, the heavy chain and light chain may be expressed from a single vector using two different promoters. In yet another alternative, the heavy chain and light chain may be expressed as a bicistronic open reading frame with the coding sequence for the two chains being separated by an IRES. In this case, a selection marker is expressed from the same vector but from a separate open reading frame. In yet another alternative, the heavy chain, light chain, and selection marker may be expressed as a tricistronic open reading frame with the coding sequences for each of the two chains and the selection marker being separated by IRES elements.

In the embodiments where the gene system expresses a cytokine, it is desirable that the level of cytokine production be auto-regulated in order to prevent secretion of toxic levels of the cytokine. One way to accomplish this is to introduce an operator site into the DNA region between the cytokine gene and its promoter in a first ORF. A second ORF is used that encodes a transcriptional repressor that binds to the operator site under the control of a promoter that is activated as a result of signaling through the cytokine’s receptor. For example, if the cytokine is IL-2, then the promoter controlling the expression of the transcriptional repressor could be a STAT transcription factor (FIG. 5). In this way, the cells can sense the cytokine in their environment and reduce their production of the cytokine when there is sufficient cytokine already present.

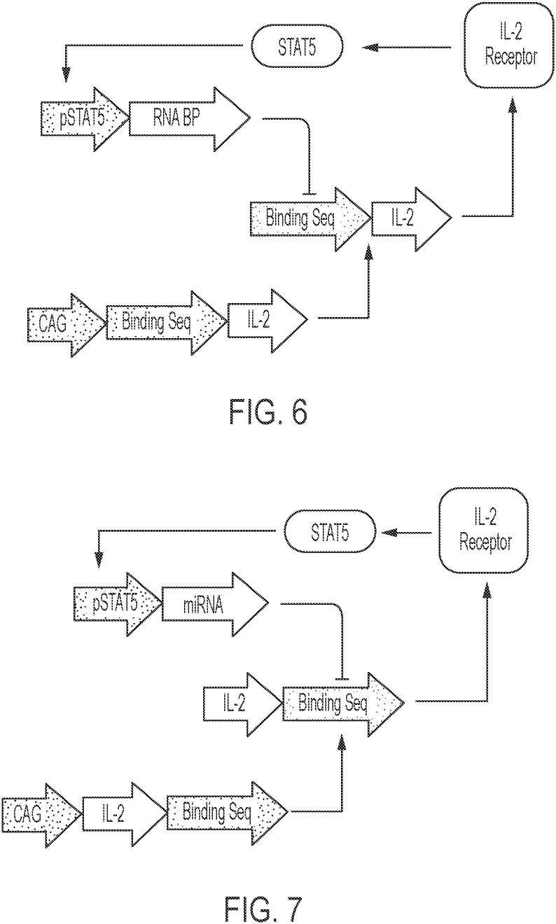

Another possible strategy is to introduce a sequence that forms a higher-order structure into the 5′ untranslated region (5′ UTR) of the cytokine gene. Then a second ORF is used that encodes an RNA-binding protein that binds to the higher-order structure, and suppresses translation, under the control of a promoter that is activated as a result of signaling through the cytokine’s receptor. For example, if the cytokine is IL-2, then the promoter controlling the expression of the RNA-binding protein could be a STAT transcription factor (FIG. 6).

Another possible strategy is to introduce several repeats of a synthetic microRNA (miRNA) target site into the 3′ untranslated region (3′ UTR) of the cytokine gene. Then a second ORF is used that encodes the miRNA under the control of a promoter that is activated as a result of signaling through the cytokine’s receptor. For example, if the cytokine is IL-2, then the promoter controlling the expression of the miRNA could be a STAT transcription factor (FIG. 7).

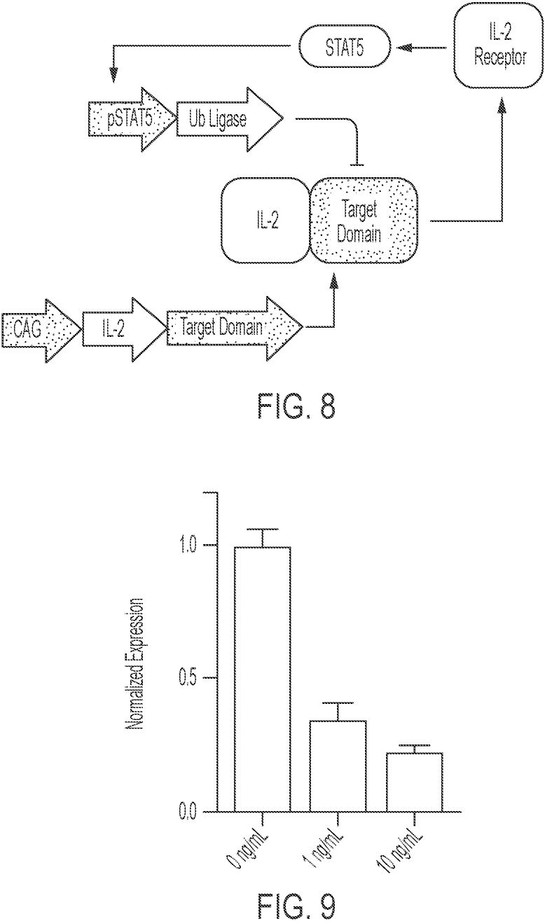

Another possible strategy is to use a second ORF encoding a synthetic ubiquitin ligase that targets the cytokine, and leads to ubiquitin-mediated proteolysis, under the control of a promoter that is activated as a result of signaling through the cytokine’s receptor. For example, if the cytokine is IL-2, then the promoter controlling the expression of the ubiquitin ligase could be a STAT transcription factor (FIG. 8). In this case, the cytokine gene may be modified to include additional protein domains if doing so is necessary in order to make the cytokine recognizable by the synthetic ubiquitin ligase. Ideally, the addition of any additional protein domains will not alter the cytokine’s immunological functions.

These self-regulated control strategies could be combined with small molecule-based strategies to provide an additional level of control to the cytokine production. Using a small molecule-activated promoter (such as the TRE/tetracycline system) to drive expression of the cytokine would allow for external regulation of the cytokine production by the administration of the small molecule. Post-transcriptional control of the cytokine expression is also possible using small molecule-dependent riboswitches - a short sequence could be added to the 5’ or 3’UTR of the cytokine gene that forms a small molecule-dependent functional higher-order structure, such as a frame-shifting aptamer or a mRNA-cleaving aptazyme, allowing for similar external control of the cytokine production, since there are examples of these systems that turn on frame-shifting or cleavage upon the addition of a small molecule and examples that turn off in the presence of the small molecule. This type of control is also possible at the protein level by adding the sequence for a destabilization domain that can be stabilized by a small molecule to the beginning or end of the gene for the cytokine, which would lead to targeted degradation of the cytokine whenever the small molecule is not present. The reverse is also possible by augmenting the gene for the cytokine with the sequence for a small molecule-assisted shutoff (SMASh) system, which includes a destabilization domain and a non-mammalian protease that cleaves the destabilization domain from the cytokine except in the presence of a small molecule protease inhibitor that would prevent cleavage and lead to degradation of the cytokine. All these modifications to the protein structure could also be done indirectly by instead modifying a synthetic transcription factor that activates the promoter controlling expression of the cytokine, which would ensure that all these protein modifications stay within the therapeutic cells instead of being secreted and potentially generating an immune response to these unnatural protein domains. One possible synthetic transcription factor to use for this purpose is a fusion between the transcriptional activators VP64, p65, and Rta (VPR) and catalytically inactivated Cas9 (dCas9), which when coexpressed with a guide RNA (gRNA) will localize the VPR complex to the synthetic promoter with complementarity to the gRNA in order to activate transcription of the cytokine gene.

The cells, of which various types are contemplated, may be encapsulated in a modified alginate core-shell. The two-layer hydrogel may be decorated with immunomodulatory small molecules to prevent an undesirable immune response. The core-shell alginate platform has a range of sizes that allow for optimal formation of the core-shell, while also maximizing nutrient access via diffusion for the cells. The core-shell may be modified to allow for timed degradation.

### I. Therapeutic Agents Expressed by Engineered Cells

An encapsulated cell composition described herein may contain a therapeutic agent produced or secreted by a cell. A therapeutic agent may include a nucleic acid (e.g., an RNA, a DNA, or an oligonucleotide), a protein (e.g., an antibody, enzyme, cytokine, hormone, receptor), a lipid, a small molecule, a metabolic agent, an oligosaccharide, a peptide, an amino acid, an antigen. In an embodiment, the encapsulated cell composition comprises a cell or a plurality of cells that are genetically engineered to produce or secrete a therapeutic agent.

In one embodiment, the encapsulated cell composition comprises a cell producing or secreting a protein. The protein may be of any size, e.g., greater than about 100 Da, 200 Da, 250 Da, 500 Da, 750 Da, 1 KDa, 1.5 kDa, 2 kDa, 2.5 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 125 kDa, 150 kDa, 200 kDa, 200 kDa, 250 kDa, 300 kDa, 400 kDa, 500 kDa, 600 kDa, 700 kDa, 800 Da, 900 kDa, or more. In an embodiment, the protein is composed of a single subunit or multiple subunits (e.g., a dimer, trimer, tetramer, etc.). A protein produced or secreted by a cell may be modified, for example, by glycosylation, methylation, or other known natural or synthetic protein modification. A protein may be produced or secreted as a pre-protein or in an inactive form and may require further modification to convert it into an active form.

Proteins produced or secreted by a cell may include antibodies or antibody fragments, for example, an Fc region or variable region of an antibody. The antibody may be an immune checkpoint inhibitor. Exemplary antibodies include anti-PD-1, anti-PD-L1, anti-CTLA4, anti-TNFα, and anti-VEGF antibodies. An antibody may be monoclonal or polyclonal. An antibody may be a nanobody. Exemplary antibody and nanobody sequences are provided in Table A.

Immune checkpoints either turn up a signal (e.g., co-stimulatory molecules) or turn down a signal. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumour necrosis factor receptor-related protein (GITR), HLA-DRB1, ICOS (also known as CD278), HLA-DQA1, HLA-E, indoleamine 2,3-dioxygenase 1 (IDOI), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Pat. Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP- 224. Nivolumab, also known as MDX-1106-04, MDX-1106, ONO-4538, BMS-936558, and OPDIVO®, is an anti-PD-1 antibody described in W02006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX- 010, MDX- 101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5844905, 5885796 and International Patent Application Nos. WO1995001994 and WO1998042752; all incorporated herein by reference, and immunoadhesins such as described in U.S. Pat. No. 8329867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is indoleamine 2,3-dioxygenase (IDO). The complete protein sequence of human IDO has Genbank accession number NP_002155. In some embodiments, the immune checkpoint inhibitor is a small molecule IDO inhibitor. Exemplary small molecules include BMS-986205, epacadostat (INCB24360), and navoximod (GDC-0919).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Another immune checkpoint protein that can be targeted in the methods provided herein is OX40, also known as CD134. The complete protein sequence of human OX40 has Genbank accession number NP_003318. In some embodiments, the immune checkpoint inhibitor is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another immune checkpoint protein that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immune checkpoint inhibitor is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

TABLE A

Exemplary Antibody Sequences

| | |
|---|---|
| Antibody Chain | Sequences Used |
| anti-PD-1 (pembrolizumab) heavy chain | QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK (SEQ ID NO: 1) |
| anti-PD-1 (pembrolizumab) light chain | EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 2) |
| anti-PD-L1 (nivolumab) heavy chain | QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK(SEQ ID NO: 3) |
| anti-PD-L1 (nivolumab) light chain | EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 4) |
| anti-CTLA4 (ipilimumab) heavy chain | QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK (SEQ ID NO: 5) |
| anti-CTLA4 (ipilimumab) light chain | EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 6) |
| anti-TNFa (adalimumab) heavy chain | MGVKVLFALICIAVAEAEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK (SEQ ID NO: 7) |
| anti-TNFa (adalimumab) light chain | DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 8) |
| anti-VEGF (bevacizumab) heavy chain | EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK (SEQ ID NO: 9) |
| anti-VEGF (bevacizumab) light chain | DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 10) |
| anti-PD-1 nanobody (CN107474135) | QVQLQESGGGLVQPGGSLRLSCAASGFTSRNYAMTWVRQAPEKGLEWVSSISSDDDSTYYEYSVKGRFTISRDNAKNTLYLQLNSLKTEDTAMYYCTKEFVAVVPVLKLGRPRDLGQGTQVTVSSAA (SEQ ID NO: 11) |
| anti-PD-L1 nanobody (Zhang et al., 2017) | QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVS (SEQ ID NO: 12) |
| anti-CTLA4 nanobody (Wan et al., 2018) | QVQLQESGGGSVQAGGSLRLSCTASGFGVDGTDMGWYRQAPGNECELVSSISSIGIGYYSESVKGRFTISRDNAKNTVYLQMNSLRPDDTAVYYCGRRWIGYRCGNWGRGTQVTVSS (SEQ ID NO: 13) |
| anti-TNFa nanobody (Efimov et al., 2016) | MGSQVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSSAGA (SEQ ID NO:14) |
| anti-VEGF nanobody (Kazemi-Monedasht et al., 2015) | QVQLQESGGGSLQAGASLRLSCAASGFAYSTYSMGWFRQVSGKEREGVATINSGTFRLWYTDSVKGSFTISRDNAKNMLYLQMNSLKPEDTAIYYCAARAWSPYSSTVDAGDFRYWGQGTQVTVSS (SEQ ID NO: 15) |

A protein produced or secreted by a cell may include a cytokine. A cytokine may be a pro-inflammatory cytokine or an anti-inflammatory cytokine. Examples of cytokines include IL-1, IL-1α, IL-1β, IL-1RA, IL-2, IL-4, IL-5, IL-6, IL-7, IL-8, IL-9, IL-10, IL-11, IL-12, IL-12a, IL-12b, IL-13, IL-14, IL-16, IL-17, G-CSF, GM-CSF, IL-20, IFN-α, IFN-β, IFN-γ, CD154, LT-β, CD70, CD153, CD178, TRAIL, TNF-α, TNF-β, SCF, M-CSF, MSP, 4-1BBL, LIF, OSM, and others. For example, a cytokine may include any cytokine described in M.J. Cameron and D.J. Kelvin, Cytokines, Chemokines, and Their Receptors (2013), Landes Biosciences, which is incorporated herein by reference in its entirety. Exemplary cytokine sequences are provided in Table B.

TABLE B

Exemplary Cytokine Sequences

| | |
|---|---|
| Cytokine Name | Sequences Used |
| mIL-12a | MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA (SEQ ID NO: 16) |
| mIL-12b | MCPQKLTISWFAIVLLVSPLMAMWELEKDVYWEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS (SEQ ID NO: 17) |
| mIL-6 | MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT (SEQ ID NO: 18) |
| mIL-4 | MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS (SEQ ID NO: 19) |
| hIL-6 | MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM (SEQ ID NO: 20) |
| hIL-4 | MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS (SEQ ID NO: 21) |
| hIL-12A | MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS (SEQ ID NO: 22) |
| hIL-12B | MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS (SEQ ID NO: 23) |
| hIL-10 | MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN (SEQ ID NO: 24) |
| mIL-10 | MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS (SEQ ID NO: 25) |
| hIL-2 | MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT (SEQ ID NO: 26) |
| mIL-7 | MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI (SEQ ID NO: 27) |
| hIL-7 | MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH (SEQ ID NO: 28) |
| hIL-15 | MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS (SEQ ID NO: 29) |
| mIL-15 | MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS (SEQ ID NO: 30) |
| hIL-2 | MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT (SEQ ID NO: 31) |
| Igis_Sig/mIL-2 | METDTLLLWVLLLWVPGSTGDMYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ (SEQ ID NO: 32) |
| mIL-2 | MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ (SEQ ID NO: 33) |
| hIL-2 | MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT (SEQ ID NO: 34) |
| hyPBase | MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF (SEQ ID NO: 35) |

An encapsulated cell composition may comprise a cell expressing a single type of therapeutic agent, e.g., a single protein or nucleic acid, or may express more than one type of therapeutic agent, e.g., a plurality of proteins or nucleic acids. In an embodiment, an implantable construct comprises a cell expressing two types of therapeutic agents (e.g., two types of proteins or nucleic acids).

In an embodiment, an encapsulated cell composition comprises a cell expressing a single type of protein, or may express more than one type of protein, e.g., a plurality of proteins. In an embodiment, an encapsulated cell composition comprises a cell expressing two types of proteins.

In an embodiment, an encapsulated cell composition comprises a cell expressing a single type of antibody or antibody fragment or may express more than one type of antibody or antibody fragment, e.g., a plurality of antibodies or antibody fragments. In an embodiment, an encapsulated cell composition comprises a cell expressing two types of antibodies or antibody fragments. In an embodiment, an encapsulated cell composition comprises a cell expressing three types of antibodies or antibody fragments. In an embodiment, an encapsulated cell composition comprises a cell expressing four types of antibodies or antibody fragments.

In an embodiment, an encapsulated cell composition comprises a cell expressing a single type of cytokine or may express more than one type of cytokine, e.g., a plurality of cytokines. In an embodiment, an encapsulated cell composition comprises a cell expressing two types of cytokines. In an embodiment, an encapsulated cell composition comprises a cell expressing three types of cytokines. In an embodiment, an encapsulated cell composition comprises a cell expressing four types of cytokines.

### II. Vector Systems for Generating Engineered Cells

One of skill in the art would be well-equipped to construct a vector through standard recombinant techniques. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors.

In particular, the pcDNA3.1, lentivirus, and Piggybac expression systems can be used to express monoclonal antibodies, nanobodies, and cytokines in mammalian cells, such as Chinese hamster ovary (CHO) cells, human embryonic kidney (HEK) cells, retinal pigmented epithelium (ARPE-10) cells, mesenchymal stem cells (MSC), human umbilical vein endothelial cells (HUVECs), murine myeloma NS0 and Sp2/0 cells, BABL/3T3 cells, MDCK cells, and PER.C6 cells, for example. All vectors can be sequence verified using Sanger sequencing.

### A. Expression Vectors

In some cases, a mammalian expression vector may be used, such as a vector designed for high-level, constitutive expression in a variety of cell types. For example, the pcDNA3.1 vector is a plasmid having a CMV promoter operably linked to the coding sequence of the molecule of interest, a BGH polyA signal, and a neomycin resistance gene for mammalian selection. Constructs having the pcDNA3.1 backbone can be transformed in DH5α Escherichia coli competent cells.

### B. Transposon Systems

Transposons, such as piggyBac, are widely used for genome engineering by insertional mutagenesis and transgenesis in a wide variety of organisms. A piggyBac transposon is bound by a transposase and contains a pair of repeat sequences. In certain embodiments, the first repeat is typically located upstream to the nucleic acid expression cassette and the second repeat is typically located downstream of the nucleic acid expression cassette. Accordingly, the second repeat represents the same sequence as the first repeat, but shows an opposite reading direction as compared with the first repeat (5′ and 3′ ends of the complementary double strand sequences are exchanged). These repeats are then termed “inverted repeats” (IRs), due to the fact that both repeats are just inversely repeated sequences. In certain embodiments, repeats may occur in a multiple number upstream and downstream of the above-mentioned nucleic acid expression cassette. Preferably, the number of repeats located upstream and downstream of the above-mentioned nucleic acid expression cassette is identical. In certain embodiments, the repeats are short, between 10-20 base pairs, and preferably 15 base pairs.

The repeats (IRs) flank a nucleic acid expression cassette that is inserted into the DNA of a cell. The nucleic acid expression cassette can include all or part of an open reading frame of a gene (i.e., that part of a protein encoding gene), one or more expression control sequences (i.e., regulatory regions in nucleic acid) alone or together with all or part of an open reading frame. Preferred expression control sequences include, but are not limited to promoters, enhancers, border control elements, locus-control regions or silencers. In a preferred embodiment, the nucleic acid expression cassette comprises a promoter operably linked to at least a portion of an open reading frame.

The transposase may be present as a polypeptide. Alternatively, the transposase is present as a polynucleotide that includes a coding sequence encoding a transposase. The polynucleotide can be RNA, for instance an mRNA encoding the transposase, or DNA, for instance a coding sequence encoding the transposase. When the transposase is present as a coding sequence encoding the transposase, in some aspects of the invention the coding sequence may be present on the same vector that includes the transposon, i.e., in cis. In other aspects of the invention, the transposase coding sequence may be present on a second vector, i.e., in trans. In certain preferred embodiments, the transposase is a mammalian piggyBac transposase.

The transposase recognizes the transposon-specific inverted terminal repeat sequences (ITRs) located on both ends of the transposon vector, moves the contents from the original sites, and integrates them into TTAA chromosomal sites through a ‘cut’ and ‘paste’ mechanism.

Piggybac constructs can be transformed into Stbl3 E. coli competent cells.

### C. Viral Systems

In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor-mediated endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Retroviruses have promise as gene delivery vectors due to their ability to integrate their genes into the host genome, transfer a large amount of foreign genetic material, infect a broad spectrum of species and cell types, and be packaged in special cell-lines.

In order to construct a retroviral vector, a nucleic acid is inserted into the viral genome in place of certain viral sequences to produce a virus that is replication-defective. In order to produce virions, a packaging cell line containing the gag, pol, and env genes—but without the LTR and packaging components—is constructed. When a recombinant plasmid containing a cDNA, together with the retroviral LTR and packaging sequences, is introduced into a special cell line (e.g., by calcium phosphate precipitation), the packaging sequence allows the RNA transcript of the recombinant plasmid to be packaged into viral particles, which are then secreted into the culture medium. The medium containing the recombinant retroviruses is then collected, optionally concentrated, and used for gene transfer. Retroviral vectors are able to infect a broad variety of cell types. However, integration and stable expression require the division of host cells.

Lentiviruses are complex retroviruses, which, in addition to the common retroviral genes gag, pol, and env, contain other genes with regulatory or structural function. Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

Third-generation lentiviruses can be generated by seeding HEK293T cells (ATCC) and co-transfecting the cells with the plasmid encoding for the desired antibody, and the packaging plasmids pMLg/PRRE (Addgene plasmid #12251), pRSV-Rev (Addgene plasmid #12253) and pMD2.g (Addgene plasmid # 12259) in a 2:5:2.5:3 ratio, respectively, using JetPrime (Polyplus transfection). The medium is replaced with fresh medium 8 h post-transfection and the virus-containing medium is collected after 48 h. The virus is concentrated using a Lenti-X concentrator (Clontech) according to the manufacturer’s protocol. To generate antibody-expressing stable cell lines, HEK293 cells are transduced with the respective virus and selected for two weeks with 1 mg/ml geneticin. Following selection with the antibiotic, sorting is performed to collect the cells expressing the highest amount of antibody.

Lentiviral constructs can be transformed into Stbl3 E. coli competent cells.

### III. Characterization and Purification of Antibodies

Encapsulated cells can be cultured and supernatants assayed for target protein production levels.

### A. Purification of Antibodies

The antibodies can be purified using the HiTrap MabSelect SuRe (GE Healthcare), according to the manufacturer’s protocol. These columns are pre-packed with Mab Select, which is a bioprocess resin for capturing of mAbs from large sample volumes.

### B. Enzyme Linked Immunosorbent Assay (ELISA)

The amount of antibody secreted by the cells can be quantified using an ELISA kit (Invitrogen, Catalog # 991000) according to the manufacturer’s protocol. ELISA kits are specific to the clone of the antibody being produced, and can be a sandwich format to increase sensitivity.

### C. PD-⅟PD-L1 Blockade Assay

The potency and stability of anti-PD-1 and anti-PD-L1 antibodies expressed in the aforementioned mammalian cell lines can be measured by using the PD-⅟PD-L1 blockade bioassay (Catalog # J1250, Promega) according to the manufacturer’s protocol. This assay, which consists of two genetically engineered cells lines, measures the ability of biologics to block immune checkpoint signals and the potency and stability of antibodies designed to block the PD-⅟PD-L1 interaction.

### D. CTLA Blockade Assay

The potency and stability of anti-CTLA4 antibodies expressed in the aforementioned mammalian cell lines can be measured by using the CTLA4 blockade bioassay (Catalog # JA3001, Promega) according to the manufacturer’s protocol. This assay is very similar to the PD-⅟PD-L1 described above, except that it reflects the mechanism of action of biologics designed to block the interaction of CTLA-4 with its ligands, CD80 and CD86.

### E. Western Blot

Western blot analysis of the heavy chain and light chain polypeptides secreted from the cells expressing the different plasmid constructs can be performed under reducing and non-reducing conditions (Ho et al., 2012). Proteins can be digested and run on a gel, followed by quantification with antibodies targeting the heavy and light chains.

### F. Evaluation of Antibody-Specific Productivity

Antibody-specific productivity will be calculated using the equation:

$${q}_{m A b} = \frac{{m}_{m A b}}{\frac{\left(N - {N}_{0}\right) \times t}{{\text{log}}_{e} \left(N / {N}_{0}\right)}}$$

where, m_{mAb} represents that secreted antibody, N₀ represents the initial viable cell values, N represents the final viable cell values, and t represents the days in culture (Chusainow et al., 2009).

### G. Glycosylation Pattern Analysis

The glycosylation pattern of the purified monoclonal antibodies can be analyzed using matrix-assisted laser desorption ionization-time of flight mass spectrometry, according to the previously described protocol (Ho et al., 2012). Characterization of glycoproteins involves identification of glycosylation sites through peptide mapping, determination of structure, as well as total sugar content.

### H. Aggregation Analysis

Aggregation of purified antibodies can be analyzed using size exclusion chromatography as described previously (Ho et al., 2012).

### IV. Characterization of Cytokine Activity

The biological activity of interleukins can be assessed using the CellTrace CFSE Cell Proliferation Kit (ThermoFisher Cat # C34554). This involves collecting cell supernatant containing secreted interleukins, followed by co-culture with isolated splenocytes over a period of 7 days, while incubation with CFSE, a cell membrane dye that is used to monitor distinct generations of proliferating cells by dye dilution. At least 6 generations of cells can be identified by distinct peaks in fluorescent signal.

### V. Controlling Drug Delivery and Release Kinetics

The vector systems may further comprise a kill switch to arrest the therapy, similar to the kill switch designed for CAR T cells. Two engineered proteins will be located inside the encapsulated cells, that dimerize when exposed to a small molecule drug called rimiducid. This drug activates a protein called caspase-9, which induces cell death.

Chemical Induction of Dimerization (CID) with small molecules is an effective technology used to generate switches of protein function to alter cell physiology. A high specificity, efficient dimerizer is rimiducid (AP1903), which has two identical, protein-binding surfaces arranged tail-to-tail, each with high affinity and specificity for a mutant or variant of FKBP12: FKBP12(F36V) (FKBP12v36, FV36 or FV). Attachment of one or more FV domains onto one or more cell signaling molecules that normally rely on homodimerization can convert that protein to rimiducid control. For example, a molecular switch is provided that provides the option to activate a pro-apoptotic polypeptide, such as, for example, Caspase-9, with rimiducid, wherein the chimeric pro-apoptotic polypeptide comprises a rimiducid-induced switch.

In one embodiment of the switch technology, a homodimerizer, such as AP1903 (rimiducid), activates a safety switch, causing apoptosis of the modified cell. In this embodiment, for example, a chimeric pro-apoptotic polypeptide, such as, for example, Caspase-9, comprising a FKBP12 multimerizing region is expressed in a cell. Upon contacting the cell with a dimerizer that binds to the Fv regions, the chimeric polypeptide dimerizes or multimerizes, and activates the cell. The cell may, for example, be an engineered cell that expresses an antibody or cytokine.

In addition, neoantigens can be introduced into the cell surface thereby marking the cells for destruction in the event of cell escape from the capsule or capsule degradation.

Furthermore, a transmembrane sensor can be engineered into the cytokine-secreting cells to create a feedback loop to regulate cytokine output. The transmembrane sensor responds to varying concentrations of the protein of interest and uses a negative feedback loop to suppress the transcription of the cytokine of interest, with the help of an inducible promoter. This allows fine-tuning of the localized delivery of the protein of interest and ensures that there is no over-expression of the protein of interest. The alginate biomaterial used allows for rapid diffusion across the inner and outer shell to give real-time feedback to this sense-and-respond genetic cellular circuit.

In another embodiment of the switch technology, the production of a cytokine from a cytokine-producing cell (e.g., an IL-2 producing RPE cell) is regulated in response to the level of a second component. For example, the second component may be a protein, such as interferon-y (IFN-γ). The IFN-γ may be produced locally by tumor cells in a subject as therapy is achieved. In some embodiments, destruction of the cytokine-producing cell (e.g., the IL-2 producing RPE cell) is achieved upon detection of the second component (e.g., IFN-γ). In some embodiments, the level of a cytokine (e.g., IL-2) from the cytokine-producing cell stays constant or increases until detection of the second component (e.g., IFN-γ). In some embodiments, the cytokine-producing cell is engineered to activate the apoptotic pathway upon detection of the second component (e.g., IFN-γ). Interfacing the apoptotic pathway with detection of the second component in this feedback loop may provide control over the sensitivity and response time of the implantable element.

In some embodiments, an algorithm (e.g., predictive modeling) is used to predict certain features of this feedback loop. For example, the time delay between detection of the second component (e.g., IFN-γ) and initiation of the apoptotic pathway may vary in length.

In some embodiments, control of the feedback loop comprises expression of a transcriptional repressor in response to a target gene. In some embodiments, the transcriptional repressor is EKRAB. In some embodiments, the target gene is an IFN-γ response gene (e.g., RPE65). In some embodiments, a pro-apoptotic gene is expressed under control of the transcriptional repressor. In some embodiments, the pro-apoptotic gene is bax.

### VI. Cell Encapsulation Using Core-Shell Alginate Hydrogels

Disclosure concerning cell encapsulation materials and methods can be found at least in U.S. Pat. No. 9,555,007; U.S. Pat. Publn. 2019/0184067; U.S. Pat. Publn. 2017/0355799; U.S. Pat. Publn. 2016/0280827; and PCT Publn. WO2019/067766, each of which is incorporated herein by reference in its entirety.

“Hydrogel” refers to a substance formed when an organic polymer (natural or synthetic) is cross-linked via covalent, ionic, or hydrogen bonds to create a three-dimensional open-lattice structure which entraps water molecules to form a gel. Biocompatible hydrogel refers to a polymer forms a gel which is not toxic to living cells, and allows sufficient diffusion of oxygen and nutrients to the entrapped cells to maintain viability.

“Alginate” is a collective term used to refer to linear polysaccharides formed from β-D-mannuronate and α-L-guluronate in any M/G ratio, as well as salts and derivatives thereof. The term “alginate”, as used herein, encompasses any polymer having the structure shown below, as well as salts thereof.

“Biocompatible” generally refers to a material and any metabolites or degradation products thereof that are generally non-toxic to the recipient and do not cause any significant adverse effects to the subject.

“Biodegradable” generally refers to a material that will degrade or erode by hydrolysis or enzymatic action under physiologic conditions to smaller units or chemical species that are capable of being metabolized, eliminated, or excreted by the subject. The degradation time is a function of polymer composition and morphology.

“Anti-inflammatory drug” refers to a drug that directly or indirectly reduces inflammation in a tissue. The term includes, but is not limited to, drugs that are immunosuppressive. The term includes anti-proliferative immunosuppressive drugs, such as drugs that inhibit the proliferation of lymphocytes.

“Immunosuppressive drug” refers to a drug that inhibits or prevents an immune response to a foreign material in a subject. Immunosuppressive drug generally act by inhibiting T-cell activation, disrupting proliferation, or suppressing inflammation. A person who is undergoing immunosuppression is said to be immunocompromised.

“Mammalian cell” refers to any cell derived from a mammalian subject suitable for transplantation into the same or a different subject. The cell may be xenogeneic, autologous, or allogeneic. The cell can be a primary cell obtained directly from a mammalian subject. The cell may also be a cell derived from the culture and expansion of a cell obtained from a subject. For example, the cell may be a stem cell. Immortalized cells are also included within this definition. In some embodiments, the cell has been genetically engineered to express a recombinant protein and/or nucleic acid.

“Autologous” refers to a transplanted biological substance taken from the same individual.

“Allogeneic” refers to a transplanted biological substance taken from a different individual of the same species.

“Xenogeneic” refers to a transplanted biological substance taken from a different species.

“Transplant” refers to the transfer of a cell, tissue, or organ to a subject from another source. The term is not limited to a particular mode of transfer. Encapsulated cells may be transplanted by any suitable method, such as by injection or surgical implantation.

### A. Biocompatible Polymers for Encapsulating Cells

The disclosed compositions are formed from a biocompatible, hydrogel-forming polymer encapsulating the cells to be transplanted. Examples of materials which can be used to form a suitable hydrogel include polysaccharides such as alginate, collagen, chitosan, sodium cellulose sulfate, gelatin and agarose, water soluble polyacrylates, polyphosphazines, poly(acrylic acids), poly(methacrylic acids), poly(alkylene oxides), poly(vinyl acetate), polyvinylpyrrolidone (PVP), and copolymers and blends of each. See, for example, U.S. Pat. Nos. 5,709,854, 6,129,761, and 6,858,229.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups and sulfonic acid groups.

Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

The biocompatible, hydrogel-forming polymer is preferably a water-soluble gelling agent. In preferred embodiments, the water-soluble gelling agent is a polysaccharide gum, more preferably a polyanionic polymer.

The engineered cells are preferably encapsulated using an anionic polymer such as alginate to provide the hydrogel layer (e.g., core), where the hydrogel layer is subsequently cross-linked with a polycationic polymer (e.g., an amino acid polymer such as polylysine) to form a shell. See e.g., U.S. Pat. Nos. 4,806,355, 4,689,293 and 4,673,566 to Goosen et al.; U.S. Pat. Nos. 4,409,331, 4,407,957, 4,391,909 and 4,352,883 to Lim et al.; U.S. Pat. Nos. 4,749,620 and 4,744,933 to Rha et al.; and U.S. Pat. No. 5,427,935 to Wang et al. Amino acid polymers that may be used to crosslink hydrogel forming polymers such as alginate include the cationic poly(amino acids) such as polylysine, polyarginine, polyornithine, and copolymers and blends thereof.

### 1. Polysaccharides

Several mammalian and non-mammalian polysaccharides have been explored for cell encapsulation. Exemplary polysaccharides suitable for cell encapsulation include alginate, chitosan, hyaluronan (HA), and chondroitin sulfate. Alginate and chitosan form crosslinked hydrogels under certain solution conditions, while HA and chondroitin sulfate are preferably modified to contain crosslinkable groups to form a hydrogel.

In preferred embodiments, the biocompatible, hydrogel-forming polymer encapsulating the cells is an alginate. Alginates are a family of unbranched anionic polysaccharides derived primarily from brown algae which occur extracellularly and intracellularly at approximately 20% to 40% of the dry weight. The 1,4-linked α-1-guluronate (G) and β-d-mannuronate (M) are arranged in homopolymeric (GGG blocks and MMM blocks) or heteropolymeric block structures (MGM blocks). Cell walls of brown algae also contain 5% to 20% of fucoidan, a branched polysaccharide sulphate ester with I-fucose four-sulfate blocks as the major component. Commercial alginates are often extracted from algae washed ashore, and their properties depend on the harvesting and extraction processes.

Alginate forms a gel in the presence of divalent cations via ionic crosslinking. Although the properties of the hydrogel can be controlled to some degree through changes in the alginate precursor (molecular weight, composition, and macromer concentration), alginate does not degrade, but rather dissolves when the divalent cations are replaced by monovalent ions. In addition, alginate does not promote cell interactions.

A particularly preferred composition is a microcapsule containing cells immobilized in a core of alginate with a polylysine shell. Preferred microcapsules may also contain an additional external alginate layer (e.g., envelope) to form a multi-layer alginate/polylysine-alginate/alginate-cells microcapsule. See U.S. Pat. No. 4,391,909 to Lim et al. for description of alginate hydrogel crosslinked with polylysine. Other cationic polymers suitable for use as a cross-linker in place of polylysine include poly(β-amino alcohols) (PBAAs) (Ma M, et al. Adv. Mater. 23:H189-94 (2011).

Chitosan is made by partially deacetylating chitin, a natural nonmammalian polysaccharide, which exhibits a close resemblance to mammalian polysaccharides, making it attractive for cell encapsulation. Chitosan degrades predominantly by lysozyme through hydrolysis of the acetylated residues. Higher degrees of deacetylation lead to slower degradation times, but better cell adhesion due to increased hydrophobicity. Under dilute acid conditions (pH<6), chitosan is positively charged and water soluble, while at physiological pH, chitosan is neutral and hydrophobic, leading to the formation of a solid physically crosslinked hydrogel. The addition of polyol salts enables encapsulation of cells at neutral pH, where gelation becomes temperature dependent.

Chitosan has many amine and hydroxyl groups that can be modified. For example, chitosan has been modified by grafting methacrylic acid to create a crosslinkable macromer while also grafting lactic acid to enhance its water solubility at physiological pH. This crosslinked chitosan hydrogel degrades in the presence of lysozyme and chondrocytes. Photopolymerizable chitosan macromer can be synthesized by modifying chitosan with photoreactive azidobenzoic acid groups. Upon exposure to UV in the absence of any initiator, reactive nitrene groups are formed that react with each other or other amine groups on the chitosan to form an azo crosslink.

Hyaluronan (HA) is a glycosaminoglycan present in many tissues throughout the body that plays an important role in embryonic development, wound healing, and angiogenesis. In addition, HA interacts with cells through cell-surface receptors to influence intracellular signaling pathways. Together, these qualities make HA attractive for tissue engineering scaffolds. HA can be modified with crosslinkable moieties, such as methacrylates and thiols, for cell encapsulation. Crosslinked HA gels remain susceptible to degradation by hyaluronidase, which breaks HA into oligosaccharide fragments of varying molecular weights. Auricular chondrocytes can be encapsulated in photopolymerized HA hydrogels where the gel structure is controlled by the macromer concentration and macromer molecular weight. In addition, photopolymerized HA and dextran hydrogels maintain long-term culture of undifferentiated human embryonic stem cells. HA hydrogels have also been fabricated through Michael-type addition reaction mechanisms where either acrylated HA is reacted with PEG-tetrathiol, or thiol-modified HA is reacted with PEG diacrylate.

Chondroitin sulfate makes up a large percentage of structural proteoglycans found in many tissues, including skin, cartilage, tendons, and heart valves, making it an attractive biopolymer for a range of tissue engineering applications. Photocrosslinked chondroitin sulfate hydrogels can be been prepared by modifying chondroitin sulfate with methacrylate groups. The hydrogel properties were readily controlled by the degree of methacrylate substitution and macromer concentration in solution prior to polymerization. Further, the negatively charged polymer creates increased swelling pressures allowing the gel to imbibe more water without sacrificing its mechanical properties. Copolymer hydrogels of chondroitin sulfate and an inert polymer, such as PEG or PVA, may also be used.

### 2. Synthetic Polymers

Polyethylene glycol (PEG) has been the most widely used synthetic polymer to create macromers for cell encapsulation. A number of studies have used poly(ethylene glycol) di(meth)acrylate to encapsulate a variety of cells. Biodegradable PEG hydrogels can be been prepared from triblock copolymers of poly(α-hydroxy esters)-b-poly (ethylene glycol)-b-poly(α-hydroxy esters) endcapped with (meth)acrylate functional groups to enable crosslinking. PLA and poly(8-caprolactone) (PCL) have been the most commonly used poly(α-hydroxy esters) in creating biodegradable PEG macromers for cell encapsulation. The degradation profile and rate are controlled through the length of the degradable block and the chemistry. The ester bonds may also degrade by esterases present in serum, which accelerates degradation.

Biodegradable PEG hydrogels can also be fabricated from precursors of PEG-bis-[2-acryloyloxy propanoate]. As an alternative to linear PEG macromers, PEG-based dendrimers of poly(glycerol-succinic acid)-PEG, which contain multiple reactive vinyl groups per PEG molecule, can be used. An attractive feature of these materials is the ability to control the degree of branching, which consequently affects the overall structural properties of the hydrogel and its degradation. Degradation will occur through the ester linkages present in the dendrimer backbone.

The biocompatible, hydrogel-forming polymer can contain polyphosphoesters or polyphosphates where the phosphoester linkage is susceptible to hydrolytic degradation resulting in the release of phosphate. For example, a phosphoester can be incorporated into the backbone of a crosslinkable PEG macromer, poly(ethylene glycol)-di-[ethylphosphatidyl (ethylene glycol) methacrylate] (PhosPEG-dMA), to form a biodegradable hydrogel. The addition of alkaline phosphatase, an ECM component synthesized by bone cells, enhances degradation. The degradation product, phosphoric acid, reacts with calcium ions in the medium to produce insoluble calcium phosphate inducing autocalcification within the hydrogel. Poly(6-aminoethyl propylene phosphate), a polyphosphoester, can be modified with methacrylates to create multivinyl macromers where the degradation rate was controlled by the degree of derivitization of the polyphosphoester polymer.

Polyphosphazenes are polymers with backbones consisting of nitrogen and phosphorous separated by alternating single and double bonds. Each phosphorous atom is covalently bonded to two side chains. The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Hydrolytically stable polyphosphazenes are formed of monomers having carboxylic acid side groups that are crosslinked by divalent or trivalent cations such as Ca2+ or Al3+. Polymers can be synthesized that degrade by hydrolysis by incorporating monomers having imidazole, amino acid ester, or glycerol side groups. Bioerodible polyphosphazines have at least two differing types of side chains, acidic side groups capable of forming salt bridges with multivalent cations, and side groups that hydrolyze under in vivo conditions, e.g., imidazole groups, amino acid esters, glycerol and glucosyl. Hydrolysis of the side chain results in erosion of the polymer. Examples of hydrolyzing side chains are unsubstituted and substituted imidizoles and amino acid esters in which the group is bonded to the phosphorous atom through an amino linkage (polyphosphazene polymers in which both R groups are attached in this manner are known as polyaminophosphazenes). For polyimidazolephosphazenes, some of the “R” groups on the polyphosphazene backbone are imidazole rings, attached to phosphorous in the backbone through a ring nitrogen atom.

### B. Immunomodulatory Exterior

An encapsulated cell composition described herein may comprise a material that reduces or inhibits a reaction (e.g., such as an immunomodulatory reaction) with or on a therapeutic agent disposed within. For example, an implantable construct comprises a zone or layer that shields a therapeutic agent from exposure to the surrounding milieu, such as host tissue, host cells, or host cell products. In an embodiment, an implantable construct minimizes the effect of a host response (e.g., an immune response) directed at a therapeutic agent disposed within, e.g., as compared with a similar therapeutic agent that is not disposed within an implantable construct.

The encapsulated cell composition may comprise a permeable, semi-permeable, or impermeable material to control the flow of solution in and out of the implantable construct. For example, the material may be permeable or semi-permeable to allow free passage of small molecules, such as nutrients and waste products, in and out of the construct. In addition, the material may be permeable or semi-permeable to allow the transport of an antigenic or therapeutic agent, out of the implantable construct. Exemplary materials include polymers, metals, ceramics, and combinations thereof.

In an embodiment, the encapsulated cell composition comprises a polymer (e.g., a naturally occurring polymer or a synthetic polymer). For example, a polymer may comprise polystyrene, polyester, polycarbonate, polyethylene, polypropylene, polyfluorocarbon, nylon, polyacetylene, polyvinyl chloride (PVC), polyolefin, polyurethane, polyacrylate, polymethacrylate, polyacrylamide, polymethacrylamide, polymethyl methacrylate, poly(2-hydroxyethyl methacrylate), polysiloxane, polydimethylsiloxane (PDMS), polyhydroxyalkanoate, PEEK®, polytetrafluoroethylene, polyethylene glycol, polysulfone, polyacrylonitrile, collagen, cellulose, cellulosic polymers, polysaccharides, polyglycolic acid, poly(L-lactic acid) (PLLA), poly(lactic glycolic acid) (PLGA), polydioxanone (PDA), poly(lactic acid), hyaluronic acid, agarose, alginate, chitosan, or a blend or copolymer thereof. In an embodiment, the implantable construct comprises a polysaccharide (e.g., alginate, cellulose, hyaluronic acid, or chitosan). In an embodiment, the encapsulated cell composition comprises alginate. In some embodiments, the average molecular weight of the polymer is from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about about 80 kDa, from about 20 kDa to about 70 kDa, from about 22.5 kDa to about 60 kDa, or from about 25 kDa to about 50 kDa). The encapsulated cell composition may comprise at least 0.5%, 1%, 2%, 3%, 4%, 5%, 7.5%, 10%, 15%, 20%, 30%, 40%, 50%, 60%, 70%, 80% or more of a polymer, e.g., a polymer described herein.

In an embodiment, the encapsulated cell composition comprises a metal or a metallic alloy. Exemplary metals or metallic alloys include titanium (e.g., nitinol, nickel titanium alloys, thermo-memory alloy materials), platinum, platinum group alloys, stainless steel, tantalum, palladium, zirconium, niobium, molybdenum, nickel-chrome, cobalt, tantalum, chromium molybdenum alloys, nickel-titanium alloys, and cobalt chromium alloys. In an embodiment, the implantable construct comprises stainless steel grade. The encapsulated cell composition may comprise at least 0.5%, 1%, 2%, 3%, 4%, 5%, 7.5%, 10%, 15%, 20%, 30%, 40%, 50%, 60%, 70%, 80% or more of a metal or metallic alloy, e.g., a metal or metallic alloy described herein.

In an embodiment, the encapsulated cell composition comprises a ceramic. Exemplary ceramics include a carbide, nitride, silica, or oxide materials (e.g., titanium oxides, hafnium oxides, iridium oxides, chromium oxides, aluminum oxides, and zirconium oxides). The encapsulated cell composition may comprise at least 0.5%, 1%, 2%, 3%, 4%, 5%, 7.5%, 10%, 15%, 20%, 30%, 40%, 50%, 60%, 70%, 80% or more of a ceramic, e.g., a ceramic described herein.

In an embodiment, the encapsulated cell composition may comprise glass. The encapsulated cell composition may comprise at least 0.5%, 1%, 2%, 3%, 4%, 5%, 7.5%, 10%, 15%, 20%, 30%, 40%, 50%, 60%, 70%, 80% or more glass.

A material within an encapsulated cell composition may be further modified, for example, with a chemical modification. For example, a material may be coated or derivatized with a chemical modification that provides a specific feature, such as an immunomodulatory or antifibrotic feature. Exemplary chemical modifications include small molecules, peptides, proteins, nucleic acids, lipids, or oligosaccharides. The encapsulated cell composition may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein.

In some embodiments, the material is chemically modified with a specific density of modifications. The specific density of chemical modifications may be described as the average number of attached chemical modifications per given area. For example, the density of a chemical modification on a material in, on, or within an implantable construct described herein may be 0.01, 0.1, 0.5, 1, 5, 10, 15, 20, 50, 75, 100, 200, 400, 500, 750, 1,000, 2,500, or 5,000 chemical modifications per square µm or square mm.

In an embodiment, the chemical modification of a material may include a linker or other attachment moiety. These linkers may include a cross-linker, an amine-containing linker, an ester-containing linker, a photolabile linker, a peptide-containing linker, a disulfide-containing linker, an amide-containing linker, a phosphoryl-containing linker, or a combination thereof. A linker may be labile (e.g., hydrolysable). Exemplary linkers or other attachment moieties is summarized in Bioconjugate Techniques (3^{rd} ed, Greg T. Hermanson, Waltham, MA: Elsevier, Inc, 2013), which is incorporated herein by reference in its entirety.

### C. Capsules

The capsules may be two- or three-layer capsules. Preferably the capsules have a mean diameter that is greater than 1 mm, preferably 1.5 mm or greater. In some embodiments, the capsules can be as large at 8 mm in diameter.

The rate of molecules entering the capsule necessary for cell viability and the rate of therapeutic products and waste material exiting the capsule membrane are selected by modulating macrocapsule permeability. Macrocapsule permeability is also modified to limit entry of immune cells, antibodies, and cytokines into the microcapsule.

It has been shown that since different cell types have different metabolic requirements, the permeability of the membrane has to be optimized based on the cell type encapsulated in the hydrogel. The diameter of the microcapsules is an important factor that influences both the immune response towards the cell capsules as well as the mass transport across the capsule membrane.

The encapsulated cell composition described herein may take any suitable shape or morphology. For example, an implantable construct may be a sphere, spheroid, tube, cord, string, ellipsoid, disk, cylinder, sheet, torus, cube, stadiumoid, cone, pyramid, triangle, rectangle, square, or rod. An encapsulated cell composition may comprise a curved or flat section. In an embodiment, an encapsulated cell composition may be prepared through the use of a mold, resulting in a custom shape.

The encapsulated cell composition may vary in size, depending, for example, on the use or site of implantation. For example, an implantable construct may have a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an encapsulated cell composition may have a section or region with a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller. In an embodiment, an implantable construct may have a section or region with a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller.

An encapsulated cell composition comprises at least one zone capable of preventing exposure of an enclosed therapeutic agent from the outside milieu, e.g., a host effector cell or tissue. In an embodiment, the encapsulated cell composition comprises an inner zone (IZ). In an embodiment, the encapsulated cell composition comprises an outer zone (OZ). In an embodiment, either the inner zone (IZ) or outer zone (OZ) may be erodible or degradable. In an embodiment, the inner zone (IZ) is erodible or degradable. In an embodiment, the outer zone (OZ) is erodible or degradable. In an embodiment, the encapsulated cell composition comprises both an inner zone (IZ) and an outer zone (OZ), either of which may be erodible or degradable. In an embodiment, the encapsulated cell composition comprises both an inner zone (IZ) and an outer zone (OZ), wherein the outer zone is erodible or degradable. In an embodiment, the encapsulated cell composition comprises both an inner zone (IZ) and an outer zone (OZ), wherein the inner zone is erodible or degradable. The thickness of either of the zone, e.g., either the inner zone or outer zone, may be correlated with the length or duration of a “shielded” phase, in which the encapsulated therapeutic agent is protected or shielded from the outside milieu, e.g., a host effector cell or tissue.

The zone (e.g., the inner zone or outer zone) of the encapsulated cell composition may comprise a degradable entity, e.g., an entity capable of degradation. A degradable entity may comprise an enzyme cleavage site, a photolabile site, a pH-sensitive site, or other labile region that can be eroded or comprised over time. In an embodiment, the degradable entity is preferentially degraded upon exposure to a first condition (e.g., exposure to a first milieu, e.g., a first pH or first enzyme) relative to a second condition (e.g., exposure to a second milieu, e.g., a second pH or second enzyme). In one embodiment, the degradable entity is degraded at least 2, 5, 10, 20, 30, 40, 50, 60, 70, 80, or 100 times faster upon exposure to a first condition relative to a second condition. In an embodiment, the degradable entity is an enzyme cleavage site, e.g., a proteolytic site. In an embodiment, the degradable entity is a polymer (e.g., a synthetic polymer or a naturally occurring polymer, e.g., a peptide or polysaccharide). In an embodiment, the degradable entity is a substrate for an endogenous host component, e.g., a degradative enzyme, e.g., a remodeling enzyme, e.g., a collagenase or metalloprotease. In an embodiment, the degradable entity comprises a cleavable linker or cleavable segment embedded in a polymer.

In an embodiment, an encapsulated cell composition comprises a pore or opening to permit passage of an object, such as a small molecule (e.g., nutrients or waste), a protein, or a nucleic acid. For example, a pore in or on an encapsulated cell composition may be greater than 0.1 nm and less than 10 µm. In an embodiment, the implantable construct comprises a pore or opening with a size range of 0.1 µm to 10 µm, 0.1 µm to 9 µm, 0.1 µm to 8 µm, 0.1 µm to 7 µm, 0.1 µm to 6 µm, 0.1 µm to 5 µm, 0.1 µm to 4 µm, 0.1 µm to 3 µm, 0.1 µm to 2 µm.

An encapsulated cell composition described herein may comprise a chemical modification in or on any enclosed material. Exemplary chemical modifications include small molecules, peptides, proteins, nucleic acids, lipids, or oligosaccharides. The implantable construct may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein. An encapsulated cell composition may be partially coated with a chemical modification, e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated with a chemical modification.

In an embodiment, the encapsulated cell composition is formulated such that the duration of release of the therapeutic agent is tunable. For example, an encapsulated cell composition may be configured in a certain manner to release a specific amount of a therapeutic agent over time, e.g., in a sustained or controlled manner. In an embodiment, the encapsulated cell composition comprises a zone (e.g., an inner zone or an outer zone) that is degradable, and this controls the duration of therapeutic release from the construct by gradually ceasing immunoprotection of encapsulated cells or causing gradual release of the therapeutic agent.

In some embodiments, the encapsulated cell composition is chemically modified with a specific density of modifications. The specific density of chemical modifications may be described as the average number of attached chemical modifications per given area. For example, the density of a chemical modification on or in an implantable construct may be 0.01, 0.1, 0.5, 1, 5, 10, 15, 20, 50, 75, 100, 200, 400, 500, 750, 1,000, 2,500, or 5,000 chemical modifications per square µm or square mm.

An encapsulated cell composition may be formulated or configured for implantation in any organ, tissue, cell, or part of a subject. For example, the encapsulated cell composition may be implanted or disposed into the intraperitoneal space of a subject. An encapsulated cell composition may be implanted in or disposed on a tumor or other growth in a subject, or be implanted in or disposed about 0.1 mm, 0.5 mm, 1 mm, 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, 1 cm, 5 cm, 10 cm, or further from a tumor or other growth in a subject. An encapsulated cell composition may be configured for implantation, or implanted, or disposed on or in the skin, a mucosal surface, a body cavity, the central nervous system (e.g., the brain or spinal cord), an organ (e.g., the heart, eye, liver, kidney, spleen, lung, ovary, breast, uterus), the lymphatic system, vasculature, oral cavity, nasal cavity, gastrointestinal tract, bone, muscle, adipose tissue, skin, or other area.

An encapsulated cell composition may be formulated for use for any period of time. For example, an encapsulated cell composition may be used for 1 hour, 2 hours, 4 hours, 6 hours, 12 hours, 1 day, 36 hours, 2 days, 3 days, 4 days, 5 days, 6 days, 1 week, 2 weeks, 3 weeks, 4 weeks, 5 weeks, 6 weeks, 2 months, 3 months, 4 months, 5 months, 6 months, 1 year, or longer. An implantable construct can be configured for limited exposure (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). An encapsulated cell composition can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more). An encapsulated cell composition can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

### D. Cells

Encapsulated cell composition described herein may contain a cell, for example, an engineered cell. A cell be derived from any mammalian organ or tissue, including the brain, nerves, ganglia, spine, eye, heart, liver, kidney, lung, spleen, bone, thymus, lymphatic system, skin, muscle, pancreas, stomach, intestine, blood, ovary, uterus, or testes.

A cell may be derived from a donor (e.g., an allogeneic cell), derived from a subject (e.g., an autologous cell), or from another species (e.g., a xenogeneic cell). In an embodiment, a cell can be grown in cell culture, or prepared from an established cell culture line, or derived from a donor (e.g., a living donor or a cadaver). In an embodiment, a cell is genetically engineered. In another embodiment, a cell is not genetically engineered. A cell may include a stem cell, such as a reprogrammed stem cell, or an induced pluripotent cell. Exemplary cells include mesenchymal stem cells (MSCs), fibroblasts (e.g., primary fibroblasts). HEK cells (e.g., HEK293T), Jurkat cells, HeLa cells, retinal pigment epithelial (RPE) cells, HUVEC cells, NIH3T3 cells, CHO-K1 cells, COS-1 cells, COS-7 cells, PC-3 cells, HCT 116 cells, A549MCF-7 cells, HuH-7 cells, U-2 OS cells, HepG2 cells, Neuro-2a cells, and SF9 cells.

A cell included in an implantable construct may produce or secrete a therapeutic therapeutic agent. In an embodiment, a cell included in an implantable construct may produce or secrete a single type of therapeutic agent or a plurality of therapeutic agents. In an embodiment, an implantable construct may comprise a cell that is transduced or transfected with a nucleic acid (e.g., a vector) comprising an expression sequence of a therapeutic agent. For example, a cell may be transduced or transfected with a lentivirus. A nucleic acid introduced into a cell (e.g., by transduction or transfection) may be incorporated into a nucleic acid delivery system, such as a plasmid, or may be delivered directly. In an embodiment, a nucleic acid introduced into a cell (e.g., as part of a plasmid) may include a region to enhance expression of the therapeutic agent and/or to direct targeting or secretion, for example, a promoter sequence, an activator sequence, or a cell-signaling peptide, or a cell export peptide. Exemplary promoters include EF-1a, CMV, Ubc, hPGK, VMD2, and CAG.

An encapsulated cell composition described herein may comprise a cell or a plurality of cells. In the case of a plurality of cells, the concentration and total cell number may be varied depending on a number of factors, such as cell type, implantation location, and expected lifetime of the encapsulated cell composition. In an embodiment, the total number of cells included in an encapsulated cell composition is greater than about 2, 4, 6, 8, 10, 20, 30, 40, 50, 75, 100, 200, 250, 500, 750, 1000, 1500, 2000, 5000, 10000, or more. In an embodiment, the total number of cells included in an encapsulated cell composition is greater than about 1.0 x 10², 1.0 x 10³, 1.0 x 10⁴, 1.0 x 10⁵, 1.0 x 10⁶, 1.0 x 10⁷, 1.0 x 10⁸, 1.0 x 10⁹, 1.0 x 10¹⁰, or more. In an embodiment, the total number of cells included in an encapsulated cell composition is less than about than about 10000, 5000, 2500, 2000, 1500, 1000, 750, 500, 250, 200, 100, 75, 50, 40, 30, 20, 10, 8, 6, 4, 2, or less. In an embodiment, the total number of cells included in an encapsulated cell composition t is less than about 1.0 x 10¹⁰, 1.0 x 10⁹, 1.0 x 10⁸, 1.0 x 10⁷, 1.0 x 10⁶, 1.0 x 10⁵, 1.0 x 10⁴, 1.0 x 10³, 1.0 x 10², or less. In an embodiment, a plurality of cells is present as an aggregate. In an embodiment, a plurality of cells is present as a cell dispersion.

Specific features of a cell contained within an encapsulated cell composition may be determined, e.g., prior to and/or after incorporation into the implantable construct. For example, cell viability, cell density, or cell expression level may be assessed. In an embodiment, cell viability, cell density, and cell expression level may be determined using standard techniques, such as cell microscopy, fluorescence microscopy, histology, or biochemical assay.

### E. Methods of Making Capsules

Methods for encapsulating cells in hydrogels are known. In preferred embodiments, the hydrogel is a polysaccharide. For example, methods for encapsulating mammalian cells in an alginate polymer are well known and briefly described below. See, for example, U.S. Pat. No. 4,352,883 to Lim.

Alginate can be ionically cross-linked with divalent cations, in water, at room temperature, to form a hydrogel matrix. An aqueous solution containing the biological materials to be encapsulated is suspended in a solution of a water soluble polymer, the suspension is formed into droplets which are configured into discrete microcapsules by contact with multivalent cations, then the surface of the microcapsules is crosslinked with polyamino acids to form a semipermeable membrane around the encapsulated materials.

The water soluble polymer with charged side groups is crosslinked by reacting the polymer with an aqueous solution containing multivalent ions of the opposite charge, either multivalent cations if the polymer has acidic side groups or multivalent anions if the polymer has basic side groups. The preferred cations for cross-linking of the polymers with acidic side groups to form a hydrogel are divalent and trivalent cations such as copper, calcium, aluminum, magnesium, strontium, barium, and tin, although di-, tri- or tetrafunctional organic cations such as alkylammonium salts, e.g., R3N+--VVV--+NR3 can also be used. Aqueous solutions of the salts of these cations are added to the polymers to form soft, highly swollen hydrogels and membranes. The higher the concentration of cation, or the higher the valence, the greater is the degree of cross-linking of the polymer. Concentrations from as low as 0.005 M have been demonstrated to cross-link the polymer. Higher concentrations are limited by the solubility of the salt.

The preferred anions for cross-linking of polymers containing basic side chains to form a hydrogel are divalent and trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

Polyanions that can be used to form a semi-permeable membrane by reaction with basic surface groups on the polymer hydrogel include polymers and copolymers of acrylic acid, methacrylic acid, and other derivatives of acrylic acid, polymers with pendant SO3H groups such as sulfonated polystyrene, and polystyrene with carboxylic acid groups.

In a preferred embodiment, alginate capsules are fabricated from solution of alginate containing suspended cells using the encapsulator (such as an Inotech encapsulator). In some embodiments, alginates are ionically crosslinked with a polyvalent cation, such as Ca2+, Ba2+, or Sr2+. In particularly preferred embodiments, the alginate is crosslinked using BaCl2. In some embodiments, the capsules are further purified after formation. In preferred embodiments, the capsules are washed with, for example, HEPES solution, Krebs solution, and/or RPMI-1640 medium.

Cells can be obtained directly from a donor, from cell culture of cells from a donor, or from established cell culture lines. In the preferred embodiments, cells are obtained directly from a donor, washed and implanted directly in combination with the polymeric material. The cells are cultured using techniques known to those skilled in the art of tissue culture.

Cell attachment and viability can be assessed using standard techniques, such as histology and fluorescent microscopy. The function of the implanted cells can be determined using a combination of the above-techniques and functional assays. For example, in the case of hepatocytes, in vivo liver function studies can be performed by placing a cannula into the recipient’s common bile duct. Bile can then be collected in increments. Bile pigments can be analyzed by high pressure liquid chromatography looking for underivatized tetrapyrroles or by thin layer chromatography after being converted to azodipyrroles by reaction with diazotized azodipyrroles ethylanthranilate either with or without treatment with P-glucuronidase. Diconjugated and monoconjugated bilirubin can also be determined by thin layer chromatography after alkalinemethanolysis of conjugated bile pigments. In general, as the number of functioning transplanted hepatocytes increases, the levels of conjugated bilirubin will increase. Simple liver function tests can also be done on blood samples, such as albumin production. Analogous organ function studies can be conducted using techniques known to those skilled in the art, as required to determine the extent of cell function after implantation. For example, islet cells of the pancreas may be delivered in a similar fashion to that specifically used to implant hepatocytes, to achieve glucose regulation by appropriate secretion of insulin to cure diabetes. Other endocrine tissues can also be implanted.

The site, or sites, where cells are to be implanted is determined based on individual need, as is the requisite number of cells. For cells having organ function, for example, hepatocytes or islet cells, the mixture can be injected into the mesentery, subcutaneous tissue, retroperitoneum, properitoneal space, and intramuscular space.

When desired, the microcapsules may be treated or incubated with a physiologically acceptable salt such as sodium sulfate or like agents, in order to increase the durability of the microcapsule, while retaining or not unduly damaging the physiological responsiveness of the cells contained in the microcapsules. By “physiologically acceptable salt” is meant a salt that is not unduly deleterious to the physiological responsiveness of the cells encapsulated in the microcapsules. In general, such salts are salts that have an anion that binds calcium ions sufficiently to stabilize the capsule, without substantially damaging the function and/or viability of the cells contained therein. Sulfate salts, such as sodium sulfate and potassium sulfate, are preferred, and sodium sulfate is most preferred. The incubation step is carried out in an aqueous solution containing the physiological salt in an amount effective to stabilize the capsules, without substantially damaging the function and/or viability of the cells contained therein as described above. In general, the salt is included in an amount of from about 0.1 or 1 milliMolar up to about 20 or 100 millimolar, most preferably about 2 to 10 millimolar. The duration of the incubation step is not critical, and may be from about 1 or 10 minutes to about 1 or 2 hours, or more (e.g., overnight). The temperature at which the incubation step is carried out is likewise not critical, and is typically from about 4° C. up to about 37° C., with room temperature (about 21° C.) preferred.

### VII. Treatment of Diseases or Disorders

Encapsulated cells can be administered, e.g., injected or transplanted, into a patient in need thereof to treat a disease or disorder. In some embodiments, the disease is a proliferative disease. In an embodiment, the proliferative disease is cancer. A cancer may be an epithelial, mesenchymal, or hematological malignancy. A cancer includes primary malignant cells or tumors (e.g., those whose cells have not migrated to sites in the subject’s body other than the site of the original malignancy or tumor) and secondary malignant cells or tumors (e.g., those arising from metastasis, the migration of malignant cells or tumor cells to secondary sites that are different from the site of the original tumor). In an embodiment, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In an embodiment, the cancer is a fibrotic or desmoplastic solid tumor.

Exemplary cancers include carcinoma, lymphoma, blastoma, sarcoma, and leukemia or lymphoid malignancies. In an embodiment, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Other examples of cancers include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin’s Disease, Adult Hodgkin’s Lymphoma, Adult Lymphocytic Leukemia, Adult Non-Hodgkin’s Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin’s Disease, Childhood Hodgkin’s Lymphoma, Childhood Hypothalamic and Visual Pathway Glioma, Childhood Lymphoblastic Leukemia, Childhood Medulloblastoma, Childhood Non-Hodgkin’s Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing’s Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher’s Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin’s Disease, Hodgkin’s Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi’s Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin’s Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom’s Macroglobulinemia, Wilms’ Tumor, and any other hyperproliferative disease, besides neoplasia, located in an organ system listed above.

In an embodiment, the implantable construct is used to treat an autoimmune disease (e.g., diabetes, multiple sclerosis, lupus, occlusions, capsular contractions) in a subject. In some embodiments, the disease is diabetes (e.g., type 1 diabetes or type 2 diabetes). In some embodiments, the condition is fibrosis. In some embodiments, the condition is inflammation.

The implantable construct described herein may be used in a method to modulate (e.g., upregulate) the immune response in a subject. For example, upon administration to a subject, the implantable construct (or an antigenic and/or therapeutic agent disposed within) may modulate (e.g., upregulate) the level of a component of the immune system in a subject (e.g., increasing the level or decreasing the level of a component). Exemplary immune system components that may be modulated by a method described herein include T cells (e.g., an invasive T cell, a killer T cell, an effector T cell, a memory T cell, a gamma delta T cell, a helper T cell), B cells, antibodies, or other another component.

The implantable constructs described herein may further comprise an additional pharmaceutical agent, such as an anti-proliferative agent, anti-cancer agent, anti-inflammatory agent, an immunomodulatory agent, or a pain-relieving agent, e.g., for use in combination therapy. The additional pharmaceutical agent may be disposed in or on the implantable construct or may be produced by a cell disposed in or on the implantable construct. In an embodiment, the additional pharmaceutical agent is small molecule, a protein, a peptide, a nucleic acid, an oligosaccharide, or other agent.

In an embodiment, the additional pharmaceutical agent is an anti-cancer agent. In some embodiments, the anti-cancer agent is a small molecule, a kinase inhibitor, an alkylating agent, a vascular disrupting agent, a microtubule targeting agent, a mitotic inhibitor, a topoisomerase inhibitor, an anti-angiogenic agent, or an anti-metabolite. In an embodiment, the anti-cancer agent is a taxane (e.g., paclitaxel, docetaxel, larotaxel or cabazitaxel). In an embodiment, the anti-cancer agent is an anthracycline (e.g., doxorubicin). In some embodiments, the anti-cancer agent is a platinum-based agent (e.g., cisplatin or oxaliplatin). In some embodiments, the anti-cancer agent is a pyrimidine analog (e.g., gemcitabine). In some embodiments, the anti-cancer agent is chosen from camptothecin, irinotecan, rapamycin, FK506, 5-FU, leucovorin, or a combination thereof. In other embodiments, the anti-cancer agent is a protein biologic (e.g., an antibody molecule), or a nucleic acid therapy (e.g., an antisense or inhibitory double stranded RNA molecule).

### VIII. Pharmaceutical Compositions

The present disclosure features pharmaceutical compositions comprising an implantable construct comprising a zone (e.g., an inner zone and optionally an outer zone, both of which may be degradable), and a therapeutic agent, and optionally a pharmaceutically acceptable excipient. In some embodiments, the implantable construct is provided in an effective amount in the pharmaceutical composition. In some embodiments, the effective amount is a therapeutically effective amount. In some embodiments, the effective amount is a prophylactically effective amount.

Pharmaceutical compositions described herein can be prepared by any method known in the art of pharmacology. In general, such preparatory methods include the steps of bringing the implantable construct into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the implantable construct may be generally equal to the dosage of the antigenic and/or therapeutic agent which would be administered to a subject and/or a convenient fraction of such a dosage such as, for example, one-half or one-third of such a dosage.

Relative amounts of the implantable construct, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) of any component.

The implantable construct and a pharmaceutical composition thereof may be administered or implanted orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally. In an embodiment, the implantable construct is injected subcutaneously. In an embodiment, the implantable construct is injected into the intraperitoneal space. In an embodiment, the implantable construct is injected into the intraperitoneal space.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer’s solution and isotonic sodium chloride solution. In addition, sterile, fixed oils are conventionally employed as a solvent or suspending medium.

For ophthalmic use, provided compounds, compositions, and devices may be formulated as micronized suspensions or in an ointment such as petrolatum.

In an embodiment, the release of an antigenic, therapeutic, or additional pharmaceutical agent is released in a sustained fashion. In order to prolong the effect of a particular agent, it is often desirable to slow the absorption of the agent from injection. This can be accomplished by the use of a liquid suspension of crystalline or amorphous material with poor water solubility. The rate of absorption of the agent then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The implantable constructs provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific therapeutic agent employed; and like factors well known in the medical arts.

An effective amount of a therapeutic agent released from the implantable construct may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of therapeutic agent per unit dosage form (e.g., per implantable construct).

The therapeutic agent administered may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

### IX. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1 - Single Gene, Dual Vector System

As shown in FIG. 1, the heavy chain (HC) and light chain (LC) of an antibody will be expressed from two different vectors. The HC will be expressed using the human cytomegalovirus (CMV) promoter and the LC using the CMV early enhancer/chicken β actin (CAG) promoter. The vector for the HC will also express a zeocin selection marker using a weaker SV40 promoter, and the vector for the LC will also express the neomycin selection marker cassette.

### Example 2 - Single Vector, Dual Gene System

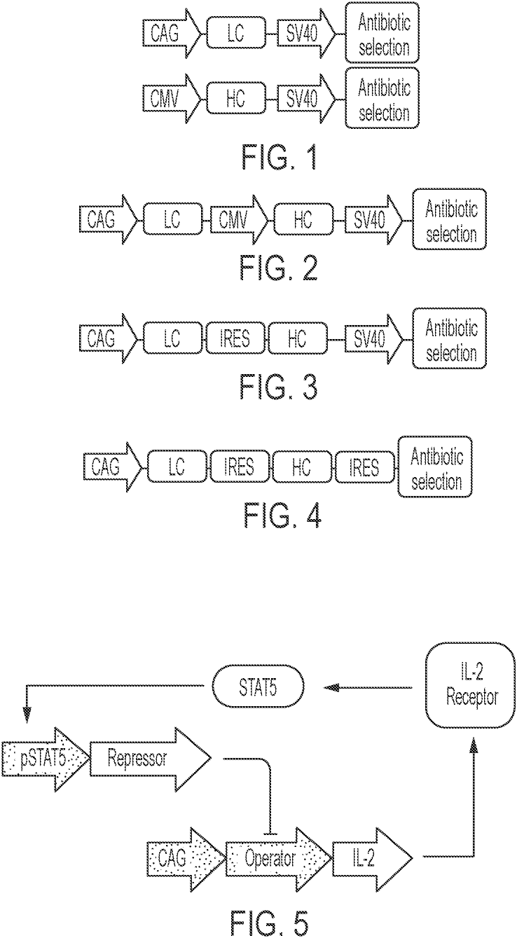

As shown in FIG. 2, the LC and HC will be expressed from a single vector using the CAG and CMV promoter, respectively. A neomycin selection marker cassette will be expressed using a weaker SV40 promoter on the same vector.

### Example 3 - Bicistronic Single ORF System

As shown in FIG. 3, the LC and HC will be expressed using the CAG promoter, separated by an internal ribosomal entry site (IRES). The weaker SV40 promoter will be used to express a neomycin selection marker cassette present on the same vector.

### Example 4 - Tricistronic Single ORF System

As shown in FIG. 4, a tricistronic vector will be generated to express the LC, HC and neomycin selection marker cassette in the same transcript mediated by two IRES under the control of the CAG promoter.

### Example 5 - Autoregulated Gene System

In the embodiments where the gene system expresses a cytokine, it is desirable that the level of cytokine production be auto-regulated in order to prevent secretion of toxic levels of the cytokine. One way to accomplish this is to introduce an operator site into the DNA region between the cytokine gene and its promoter in a first ORF. A second ORF is used that encodes a transcriptional repressor that binds to the operator site under the control of a promoter that is activated as a result of signaling through the cytokine’s receptor. For example, if the cytokine is IL-2, then the promoter controlling the expression of the transcriptional repressor could be a STAT transcription factor (FIG. 5). In this way, the cells can sense the cytokine in their environment and reduce their production of the cytokine when there is sufficient cytokine already present.

Another possible strategy is to introduce a sequence that forms a higher-order structure into the 5′ untranslated region (5′ UTR) of the cytokine gene. Then a second ORF is used that encodes an RNA-binding protein that binds to the higher-order structure, and suppresses translation, under the control of a promoter that is activated as a result of signaling through the cytokine’s receptor. For example, if the cytokine is IL-2, then the promoter controlling the expression of the RNA-binding protein could be a STAT transcription factor (FIG. 6).

Another possible strategy is to introduce several repeats of a synthetic microRNA (miRNA) target site into the 3′ untranslated region (3′ UTR) of the cytokine gene. Then a second ORF is used that encodes the miRNA under the control of a promoter that is activated as a result of signaling through the cytokine’s receptor. For example, if the cytokine is IL-2, then the promoter controlling the expression of the miRNA could be a STAT transcription factor (FIG. 7).

Another possible strategy is to use a second ORF encoding a synthetic ubiquitin ligase that targets the cytokine, and leads to ubiquitin-mediated proteolysis, under the control of a promoter that is activated as a result of signaling through the cytokine’s receptor. For example, if the cytokine is IL-2, then the promoter controlling the expression of the ubiquitin ligase could be a STAT transcription factor (FIG. 8). In this case, the cytokine gene may be modified to include additional protein domains if doing so is necessary in order to make the cytokine recognizable by the synthetic ubiquitin ligase. Ideally, the addition of any additional protein domains will not alter the cytokine’s immunological functions.

### Example 6 - Engineering Cell Lines Sensing and Reporting IFN-γ in Situ

To better elucidate in situ pharmacodynamics of therapy, a RPE cell-based sensor for the detection of Interferon γ (IFNγ)-mediated response was developed. We verified downregulation of RPE65 in RPE cells exposed to recombinant human IFNγ (FIG. 9) supporting the feasibility of our proposed study aimed at developing an RPE cell based IFNγ-response sensor that leverage detection of IFNγ transcriptional signatures and can be encapsulated in TMTD capsules for in situ monitoring therapeutic efficacy.

To develop a cellular sensor of the INFγ response amenable to pharmacodynamic studies, RPE cells were engineered to link expression of renilla luciferase gene (lux) to that of RPE65, a validated marker of IFNγ response (FIG. 9). RPE, RPE-IL-2, and RPE-IL-2-ks would then be transfected for the expression of lux under the control of the ETR operator, which is regulated by the erythromycin-dependent transrepressor (EKRAB) and the puromycin resistance gene for selection purposes. Cells will then selected and screened by monitoring luciferase signal which is constitutively expressed in the absence of EKRAB. The resulting stable cell lines will be engineered to integrate a cassette for the expression of EKRAB and the blasticidin resistance gene 3′ of RPE65 as previously shown. Cells will be selected with blasticidin and stable cell lines verified by monitoring luciferase signal upon treatment with recombinant IFNγ and/or erythromycin to verify EKRAB integration. Chromosomal integration will be validated via genomic PCR. This reporter can leverage coelenterazine as substrate, which enables the use IVIS imaging to specifically measure activity of IFNγ reporter capsules separate from signal of firefly luciferase used for tumor volume monitoring.

### Example 7 - Design of a Genetic Circuit Linking IFNγ Detection to Induction of Apoptosis

To achieve induction of apoptosis in response to detection of IFNγ response, a genetic circuit will be designed and built as described in Example 6, wherein expression of a transcriptional repressor EKRAB is linked of that of the IFNγ response target gene RPE65 (FIG. 9). In addition, the pro-apoptotic bax gene will be expressed under the control of a transcriptional repressor. As a result, the downregulation of RPE65 leads to a decrease in the expression of EKRAB and increase in the expression of BAX.

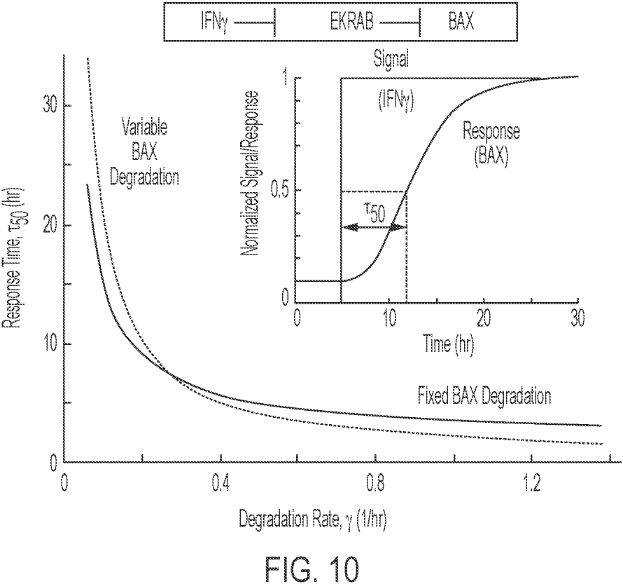

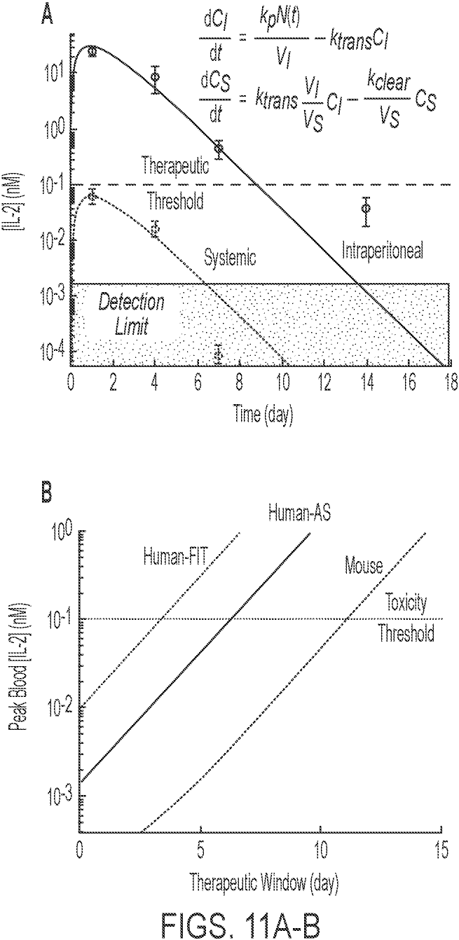

A computation model was designed in which over 10-fold activation of bax expression is predicted to be achieved in response to IFNγ, assuming 100-fold repression of EKRAB with a Hill coefficient of 2 and 5-fold repression of RPE65 (FIG. 9). The model also predicted modulating the transcriptional repressor degradation rate allows tuning the circuit response time between 3 and 30 hrs (FIG. 10). This tunable delay combined with tunable PK delay of up to 10 days at higher dosage (FIGS. 11A-B) will allow us to achieve the desired delay between IFNγ detection and therapy termination as needed based on PK/PD in vivo studies of

To achieve this goal, RPE cells expressing IL-2 (RPE/IL-2 cells) will be engineered to express the transcriptional regulator linked to RPE65. Specifically, a series of cell lines will be generated in which EKRAB or TetR linked to the expression of a fluorescent reporter (iRFP) through an internal ribosome entry site (IRES) for detection purposes and containing a blasticidin resistance gene for selection purposes will be prepared. To this end, integration cassettes containing the genes encoding EKRAB/TetR and iRFP under the control of different IRES variants will be built and fused to different degron tags to modulate half-life as previously shown. The resulting constructs will be integrated into the chromosome of RPE/IL-2 cells 3′ of RPE65, generating a series of cell lines that express EKRAB or TetR linked to the expression of RPE65, using known procedures. A modular assembly toolkit will be used that enables rapid production of large DNA cassettes through a plug-and-play approach. Cells will be selected using blasticidin and stable cell lines verified by monitoring the iRFP signal upon transient transfection for the expression of GFP under the control of EKRAB/TetR and treatment with recombinant IFNγ and/or erythromycin/tetracycline to verify EKRAB/TetR integration. Chromosomal integration will be validated via genomic PCR. Next, stable cell lines expressing EKRAB/TetR will be transfected for the expression of the proapoptotic gene bax under the control of EKRAB/TetR (ETR and TO, respectively). A cassette encoding bax linked to a fluorescent reporter (eqFP650) through a IRES and containing the puromycin resistance for selection purposes will be used linked to eqFP65 through a 2A self-cleaving peptide. Cells will be selected using puromycin and single clones expanded for selection of monoclonal populations. Stable cell lines will be verified by monitoring the eqFP650 signal and markers of early and late apoptosis (Annexin V and PI binding) upon cell exposure to recombinant IFNγ and/or erythromycin/tetracycline to validate bax expression.

The circuits will then be validated by monitoring cell fluorescence, protein levels (including IL-2 levels) using Western blot and ELISA assays, and through sequencing analyses. The relation between the concentration of IFNγ in the culturing medium, IL-2 production, and markers of early and late apoptosis will then be established. The results of these measurements will be used to refine the mathematical model of the circuit. Coupled to PK/PD model developed here, these results allow for further refinement of the design rules for the circuits predicted to result in optimal in vivo performance. These design rules will inform the selection of stable cell lines with EKRAB/TetR translational rate and degradation rate that are predicted to perform optimally in vivo.

Further, the cell lines generated in this study will be validated in vivo as described using ovarian cancer mouse models. In each of the IP cancer mouse models, groups of 10 will be implanted to ensure reproducibility and statistical significance. Initial trials will be focused on using ID8 Fluc tumors and leads will be validated using KPC and BP tumor models to ensure efficacy across tumors with various mutation burdens. For each IP tumor study, five groups of 10 mice will be used to assess anti-tumor efficacy and safety. A correlation between IL-2 dosing and time of self-destruction of IL-2-producing cells will then be determined. As such we will test 5 dosing of capsules containing the cell lines developed in the study and appropriate controls (RPE-IL2-IFNγ-KS, and sham surgical control). 3 extra mice will be injected for each group to ensure groups of 10 will have tumors of similar size. 130 C57BL/6 mice study (N = (5 experimental groups) * (n=13) = 65 mice) will be studied. Each IP cancer study will be repeated at least once to ensure reproducibility of the results. Upon conclusion of these studies, blood and IP cells and fluid will be collected for flow cytometry measurement-based immune profiling and the capsules will be explanted, imaged, and assayed for protein production using ELISA.

This study will generate sense-and-respond cellular devices that induce delayed activation of apoptosis and thus termination of therapy response to detection of IFNγ response. Integration of predictive modeling and experimental tests will allow defining the design rules of cellular control systems for optimal tuning of the apoptotic response upon detection of the desired levels of activation of the IFNγ response. These results will support the design of an in vivo platform for duration of IL-2 delivery temporally regulated to address patient-specific variability.

### Example 8 - Engineering a Cell-Based Platform for in Vivo Continuous Feedback-Regulated Delivery

To design cellular devices that that regulate IL-2 production continuously based on feedback signals generated upon detection of the IL-2 receptors, RPE cells expressing the intermediate affinity IL-2βγ receptor were engineered to repress IL-2 expression in response to STAT5 activation (which is activated by JAK-STAT signaling upon IL-2βγ receptor activation). This framework will provide a mechanism to keep IL-2 levels at concentrations required for the activation of the intermediate-affinity receptors. It was hypothesized that IL-2 expression in these cellular devices will be promptly discontinued upon accumulation of IL-2 concentrations that activate the intermediate-affinity receptors, preventing accumulation of IL-2 concentrations that result in toxicity leading to vascular leak syndrome. To this end, different circuit topologies were designed to achieve self-adjusted IL-2 production. This strategy would allow for administering larger doses of capsules or capsules with larger number of cells without the risk of immunosuppressive effects nor to reach toxic doses, leading to more robust and durable therapy regimes for patients.

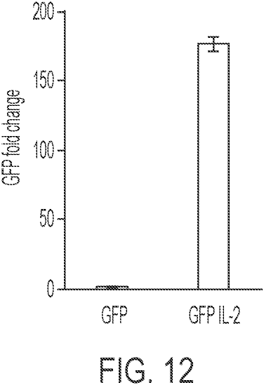

To establish feasibility of the IL-2 feedback control mechanism, control of a reporter gene (GFP) mediated by STAT5 in response to IL-2 levels was evaluated. HEK-293 cells expressing the IL-2αβγ receptor (HEK-Blue™ IL-2 cells, InvivoGen) were engineered to expresses IL-2 constitutively and GFP under a STAT5-inducible promoter. The STAT5 response elements (STAT5-RE) containing the consensus binding site for STAT5 (TTCtggGAA) was placed in tandem arrangement. Flow cytometry analyses revealed a dramatic increase in GPF signal compared to control cells lacking IL-2 (FIG. 12), demonstrating the feasibility of the approach proposed based on IL-2-mediated regulation of STAT5-dependent output.

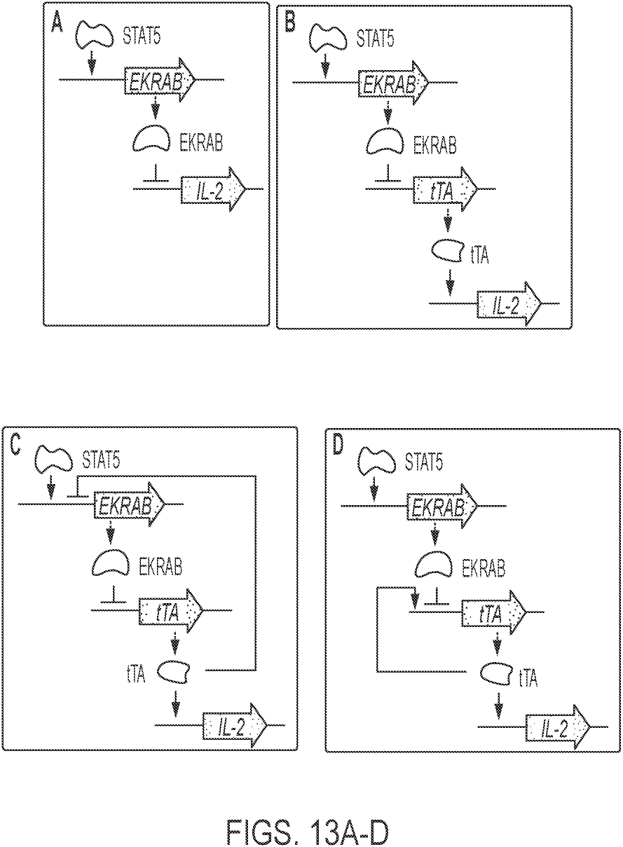

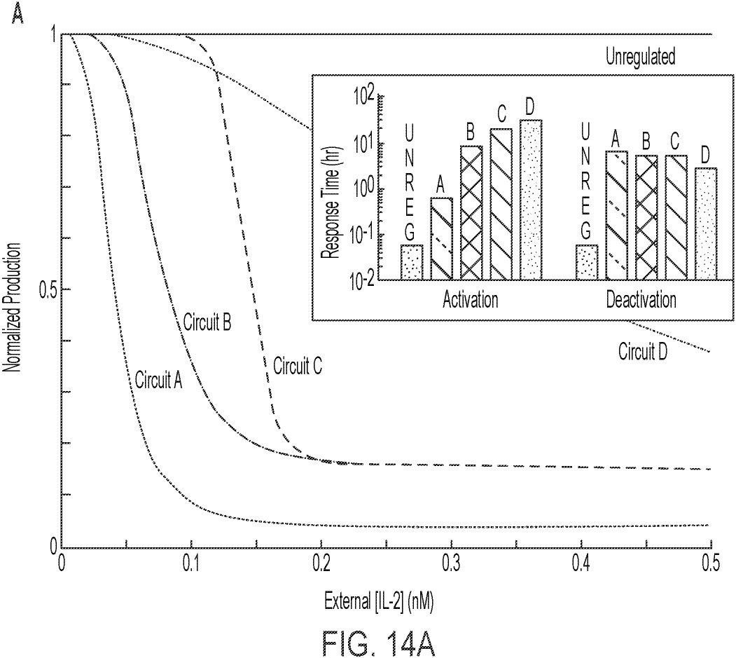

To achieve IL-2 repression in response to activation of the high affinity IL-2 receptor, four synthetic circuit topologies that execute repression of IL-2 production in response to STAT5 activation were designed, as shown in FIGS. 13A-D: (A) IL-2 is constitutively expressed under basal condition. STAT5 activates expression of EKRAB, which represses expression of IL-2; (B) IL-2 is activated by tTA under basal conditions. STAT5 activates expression of EKRAB, which represses expression of tTA; (C) IL-2 is activated by tTA and STAT5 activates expression of EKRAB. tTA and EKRAB repress each other in a toggle switch-like configuration expected to result in bistability of the system; (D) IL-2 is activated by tTA under basal conditions. STAT5 activates expression of EKRAB, which represses expression of tTA. A tTA self-amplification loop is expected to accelerate steady state production of tTA in the absence of STAT5 (under non induced conditions).

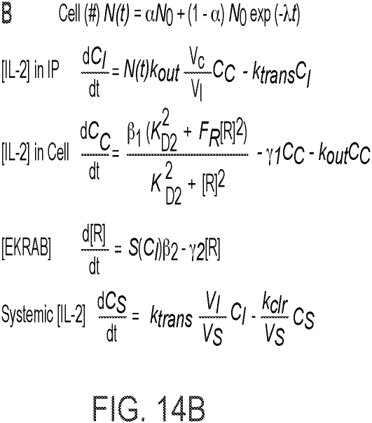

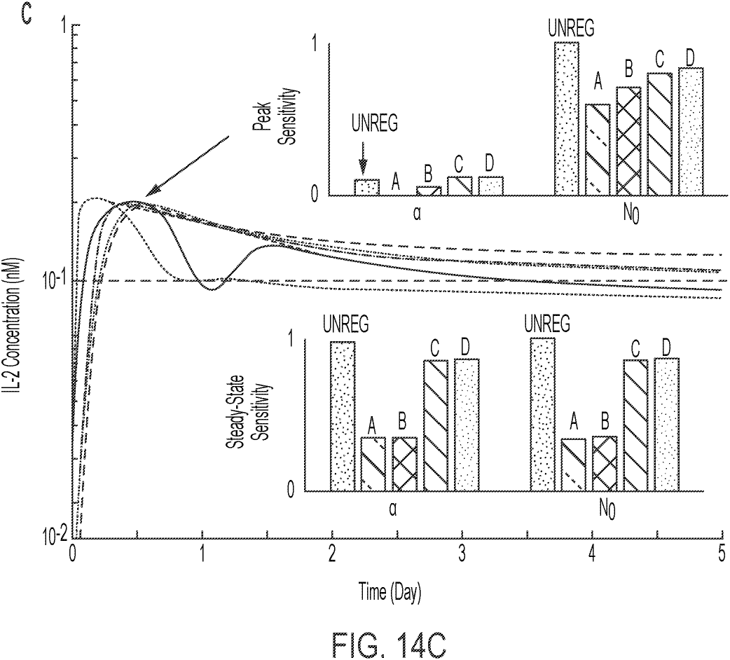

To computationally assess these designs, mathematical models were assembled that include protein-production and degradation. In the first iteration of the models, JAK-STAT signaling is modeled phenomenologically, assuming an instant response described by the Hill-equation between the level of extracellular IL-2 and the fraction of transcriptionally active STAT5. The topologies in the open-loop setting were first assessed, where it was assumed cells are placed in the environment with externally controlled IL-2 levels and changes in these IL-2 levels affect intracellular IL-2 production would be determined. The preliminary results show that different topologies lead to different open-loop dose-response curves (FIG. 14A) and differences in response times (FIG. 14A, inset). Our results indicate that the topology “A” is the fastest to respond to changes in extracellular IL-2, whereas topology “C” has the steepest dose-response curve, a feature typically indicative of robustness in the closed-loop model.

Next, the intracellular model was coupled with PK model by introducing IL-2 export flux and making IP-level of IL-2 an input for STAT signal (FIG. 14B). The results of such model obtained assuming that cell inside the capsule produce a high level of IL-2 prior to implantation, resulting in repressed initial conditions and following implantation, indicated that the IL-2 flux into the IP space will decrease IL-2 levels that cells are exposed to, leading to partial de-repression and continued IL-2 production (FIG. 14C). A sensitivity analysis indicated that all negative feedback circuits present improved robustness to changes in dosing and to the decrease of production due to cell death post-implantation (FIG. 14C, insets). Partial de-repression of IL-2 following cell death may extend the therapeutic window. These preliminary results support experimental testing of topology “A”.

To experimentally test the four circuit topologies (FIG. 13), RPE cells will be engineered to express the IL-2 signaling pathway through stable transfection of RPE cells with the human IL-2Rβ IL-2Ry genes, thereby generating cell lines that respond to IL-2 doses that result in activation of the intermediate-affinity IL-2 receptors (RPE-ILR). Stable cell lines will be validated via transient transfection with GFP under the control of STA5-responsive elements as in FIG. 12.

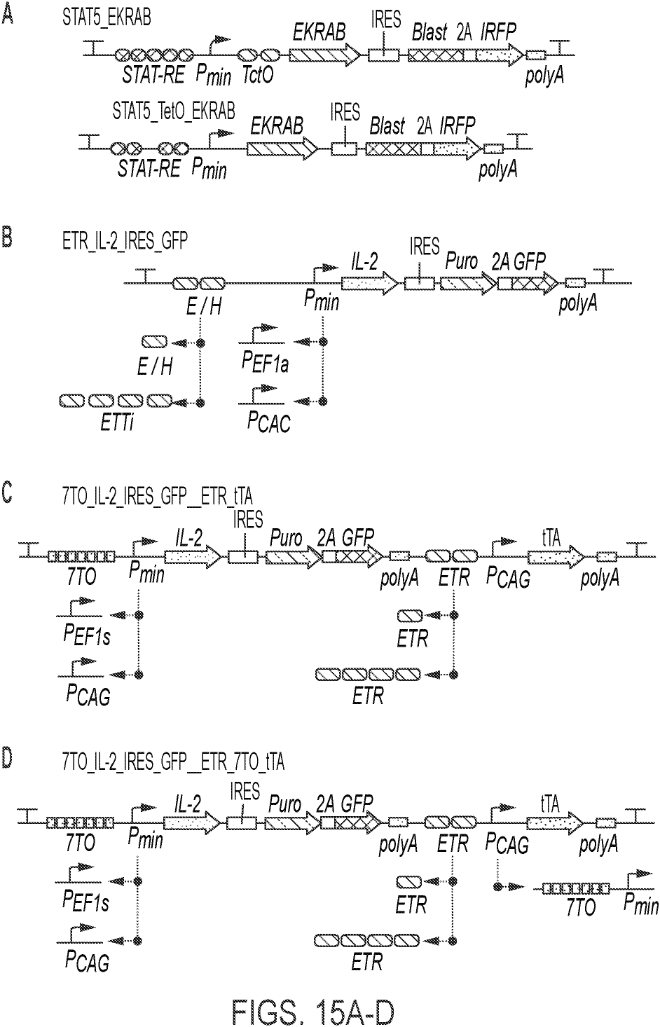

To develop and characterize IL-2 feedback control systems and fine-tune the mathematical models, RPE master cell lines that express the main regulator EKRAB as either regulated by STAT5 for building topologies A, B, and D will be generated (FIG. 15A, top) or under the control of a hybrid promoter activated by STAT5 and repressed by tTA for building topology C (FIG. 15B, bottom). The expression of EKRAB will be linked to that of a fluorescent reporter (iRFP) through an internal ribosome entry site (IRES) for detection purposes. The IRES used in this study results in a 1:3 protein expression ratio. The expression system will include a blasticidin resistance gene for selection purposes linked to iRFP through a 2A self-cleaving peptide. The resulting EKRAB expression cassettes will be integrated into the genome of RPE-IL2R cells via plasmid transfection. Cells will be selected using blasticidin and single clones expanded and screened for selection of monoclonal populations. Because preliminary modeling results pointed to the circuit components’ expression levels as relevant design parameters monoclonal populations will be screened by monitoring the iRFP signal upon transient transfection for tTA expression and treatment with recombinant IL-2 (to activate STAT5) to select cell lines displaying maximal iRFP dynamic range upon transient transfection/IL-2 treatment. The resulting monoclonal populations (STAT RE EKRAB [FIG. 15A, top] and STAT RE_TetO_EKRAB [FIG. 15A, bottom]) will be used as master cell lines for subsequent integration of the circuit components.

The master cell lines STAT RE_EKRAB and STAT RE_TetO_EKRAB will be engineered to establish a “landing pad” for rapid and facile insertion of the cassette encoding IL-2, tTA, a fluorescent reporter to monitor IL-2 expression (GFP) and the puromycin resistance gene (FIG. 14B). A dual integrase cassette exchange system (DICE) that enables genomic integration through a pair of orthogonal serine integrases will be used. First, a landing pad cassette consisting of a reporter gene (eqFP650) and a selectable marker (the zeocin resistance gene, Zeo) linked via the self-cleaving peptide 2A and under the control of the mammalian ubiquitin C (UBC) promoter will be prepared. This cassette will be flanked by the attP recognition sites for phiC31 integrase and Bxb1 integrase. CRISPR-Cas9 editing tools will be used to integrate the landing pad cassette into the AAVS1 locus, a well-established, safe harbor locus in human cells. The resulting cells will be selected with zeocin and monoclonal populations screened by flow cytometry for stable integration of the “landing pad”. Chromosomal integration will be verified via genomic PCR.

Master cell lines containing the “landing pad” will be subsequently used to generate cell lines for expression of IL-2/tTA by swapping the eqFP650 Zeo cassette with a series of cassettes containing the genes encoding IL-2/tTA from different promoter/operator variants (FIGS. 18B-D light grey) and flanked by the phiC31 and Bxb1 integrase sites. A modular assembly toolkit for rapid production of large DNA cassettes through a plug-and-play approach will be employed. Expression of the circuit components (i.e., tTA and IL-2 from different promoter/operator variants) will allow to modulate synthesis rates. Specifically, STAT RE_EKRAB cells will be transfected with “destination vectors” encoding (i) ETR_IL-2_IRES_GFP [FIG. 18B] to generate topology A, (ii) 7TO_IL-2_IRES_GFP_ETR_tTA [FIG. 14C] to generate topology B, and (iii) 7TO_IL-2_IRES_GFP_ETR_7TO_tTA [FIG. 14D] to generate topology 3. STAT RE_TetO_EKRAB cells with be transfected with “destination vectors” encoding (i) 7TO_IL-2_IRES_GFP_ETR_tTA [FIG. 14C] to generate topology 4. All transfection reactions will include a vector encoding Phi31 and Bxb1 integrases.

In addition, the circuits will be validated by monitoring cell fluorescence, protein levels (including IL-2 and IFNγ levels) using Western blot and ELISA assays, and through sequencing analyses. Correlations between the STAT5 activity (evaluated by monitoring iRFP signal) and IL-2 production (evaluated by monitoring IL-2 protein levels and GPF signal) as a function of cell number and culturing time will be made. These results will be used to refine the mathematical models. Coupled with PK model of IL-2 transport, this model will be used to formulate the design rules of robust feedback-regulated system for IL-2 production in vivo, which, in turn will guide the selection of stable cell lines with optimal circuit design and expression levels of the circuit components.

In addition, to explore the use of a cell based IL-2 delivery platform in which IL-2 expression is constantly adjusted based on IL-2 receptor-mediated feedback and the cellular devices temporally regulated based on detection of IFNγ-response, the IL-2 producing cell lines will be engineered with topology A to first integrate a cassette encoding TetR and a blasticidin resistance gene linked to iRFP through a 2A self-cleaving peptide 3′ of RPE65. The resulting cells will be selected and characterized and transfected with a plasmid encoding bax under the control of TO linked to a fluorescent reporter (eqFP650) through a IRES and the puromycin resistance for selection purposes. Cell lines containing both IL-2 mediated and IFNγ -mediated control systems will be validated by monitoring cell fluorescence, protein levels (including IL-2 levels) using Western blot and ELISA assays, as a function of small molecule inducers.

The cell therapies constructed in this aim will be validated using ovarian cancer mouse models. In each of the IP cancer mouse models, groups of 10 will be implanted to ensure reproducibility and statistical significance. Initial trials will focus on ID8 Fluc tumors, and leads will be subsequently validated using KPC and BP tumor models to ensure efficacy across tumors with various mutation burdens. It is expected that IL-2 dosing will not correlate with tumor therapy or toxicity outcomes. As such, dosing of 5 constructs and appropriate controls (RPE-IL2-REG-KS (5 doses), and sham surgical control) will be carried out. 130 C57BL/6 mice in this study (N = (5 experimental groups) * (n=13) = 65 mice). Each IP cancer study will be repeated at least once to ensure reproducibility of the results. At the conclusion of these studies, blood and IP cells and fluid will be collected for flow cytometry measurement-based immune profiling and the capsules will be explanted, imaged, and assayed for protein production using ELISA.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

CN107474135A, An anti-(PD-1) nanobody PD-⅟Nb20, and a preparing method and applications thereof. 2017: China.

Cao, J., et al., Versatile and on-demand biologics co-production in yeast. Nat Commun, 2018. 9(1): p. 77.

Chusainow, J., et al., A study of monoclonal antibody-producing CHO cell lines: what makes a stable high producer? Biotechnol Bioeng, 2009. 102(4): p. 1182-96.

DrugBank. Bevacizumab. Available from: https://www.drugbank.ca/drugs/DB00112.

Efimov, G.A., et al., Cell-type-restricted anti-cytokine therapy: TNF inhibition from one pathogenic source. Proc Natl Acad Sci USA, 2016. 113(11): p. 3006-11.

Ho, S.C., et al., IRES-mediated Tricistronic vectors for enhancing generation of high monoclonal antibody expressing CHO cell lines. J Biotechnol, 2012. 157(1): p. 130-9.

Kazemi-Lomedasht, F., et al., Inhibition of angiogenesis in human endothelial cell using VEGF specific nanobody. Mol Immunol, 2015. 65(1): p. 58-67.

Project, G. Nivolumab. Available from: https://www.genome.jp/dbgetbin/www_bget?dr:D10316.

Roybal, K.T., et al., Engineering T Cells with Customized Therapeutic Response Programs Using Synthetic Notch Receptors. Cell, 2016. 167(2): p. 419-432 e16.

Smole, A., et al., A Synthetic Mammalian Therapeutic Gene Circuit for Sensing and Suppressing Inflammation. Mol Ther, 2017. 25(1): p. 102-119.

Wan, R., et al., Screening and antitumor effect of an antiCTLA4 nanobody. Oncol Rep, 2018. 39(2): p. 511-518.

Zhang, F., et al., Structural basis of a novel PD-L1 nanobody for immune checkpoint blockade. Cell Discov, 2017. 3: p. 17004.

What is claimed is:

1. An engineered cell, or an implantable element comprising the engineered cell, wherein the engineered cell comprises an exogenous nucleic acid having a coding sequence encoding a therapeutic protein, wherein the therapeutic protein is a cytokine, wherein the cytokine coding sequence is operably linked to a repressible promoter, wherein the engineered cell further comprises at least one coding sequence encoding a transcriptional repressor that can bind to the repressible promoter, and wherein the transcriptional repressor coding sequence is operably linked to a promoter that is activated as a result of signaling through the cytokine’s receptor.

2. The engineered cell or implantable element comprising the engineered cell of claim 1, wherein the exogenous nucleic acid is integrated into a chromosome of the engineered cell.

3. The engineered cell or implantable element comprising the engineered cell of claim 1, wherein the engineered cell further comprises at least one coding sequence encoding a selection marker.

4. The engineered cell or implantable element comprising the engineered cell of claim 1, wherein the cytokine coding sequence is operably linked to a small molecule-activated promoter.

5. The engineered cell or implantable element comprising the engineered cell of claim 1, wherein the cytokine coding sequence comprises an activating or inhibiting small molecule-dependent functional higher-order structure.

6. The engineered cell or implantable element comprising the engineered cell of claim 1, wherein the cytokine coding sequence comprises a small molecule-assisted shutoff system sequence.

7. The engineered cell or implantable element comprising the engineered cell of claim 1, wherein the cytokine coding sequence is operably linked to a synthetic promoter that is activated by a synthetic transcription factor.

8. The engineered cell or implantable element comprising the engineered cell of claim 7, wherein the synthetic transcription factor comprises a catalytically inactive Cas9 (dCas9) fused to transcriptional activation domains.

9. The engineered cell or implantable element comprising the engineered cell of claim 7, wherein the synthetic transcription factor coding sequence is operably linked to a small molecule-activated promoter.

10. The engineered cell or implantable element comprising the engineered cell of claim 7, wherein the synthetic transcription factor coding sequence comprises an activating or inhibiting small molecule-dependent functional higher-order structure.

11. The engineered cell or implantable element comprising the engineered cell of claim 7, wherein the synthetic transcription factor coding sequence comprises a small molecule-assisted shutoff system sequence.

12. The implantable element comprising the engineered cell of claim 1, wherein the implantable element comprises an inner zone and an outer zone, wherein the engineered cell is present in the inner zone.

13. The implantable element comprising the engineered cell of claim 12, wherein the outer zone is configured so as to hinder contact of a host immune effector molecule or cell with the antigenic agent for an initial or shielded phase of implantation, but so as to allow contact of a host immune effector molecule or cell with the antigenic agent in a subsequent or unshielded phase of implantation.

14. The implantable element comprising the engineered cell of claim 12, wherein the outer zone comprises a degradable entity.

15. The implantable element comprising the engineered cell of claim 13, wherein the shielded phase lasts for between 0.5 days and 30 days, 1 day and 14 days, or 1 day and 7 days.

16. The implantable element comprising the engineered cell of claim 13, wherein the thickness of the outer zone correlates with the length/duration of the shielded phase.

17. The implantable element comprising the engineered cell of claim 1, wherein the implantable construct provides sustained release of the therapeutic protein.

18. The implantable element comprising the engineered cell of claim 1, wherein the implantable construct provides substantially non-pulsatile release of the therapeutic protein.

19. The implantable element comprising the engineered cell of claim 1, further comprising a polymeric hydrogel.

20. The implantable element comprising the engineered cell of claim 19, wherein the outer zone comprises a polymeric hydrogel.

21. The implantable element comprising the engineered cell of claim 19, wherein the inner zone comprises a polymeric hydrogel.

22. The implantable element comprising the engineered cell of claim 19, wherein the inner zone and the outer zone comprise the same polymeric hydrogel.

23. The implantable element comprising the engineered cell of claim 19, wherein the inner zone and the outer zone comprise two different polymeric hydrogels.

24. The implantable element comprising the engineered cell of claim 1, wherein the implantable element comprises at least about 10,000, 15,000, or 20,000 engineered cells.

25. A bioreactor comprising the engineered cell of claim 1.

26. A preparation of implantable elements comprising a plurality of implantable elements of claim 1.

27. A method of providing an implantable element to a patient, the method comprising implanting into the subject, or providing the subject with, an implantable element of claim 1.
